(19) United States
(12) Patent Application Publication
(10) Pub. No.: US 2018/0023139 A1
(43) Pub. Date: Jan. 25, 2018

(54) METHODS OF IDENTIFYING ESSENTIAL PROTEIN DOMAINS

(71) Applicant: Cold Spring Harbor Laboratory, Cold Spring Harbor, NY (US)

(72) Inventors: Christopher H. Vakoc, Cold Spring Harbor, NY (US); Junwei Shi, Cold Spring Harbor, NY (US); Justin B. Kinney, Forest Hills, NY (US)

(73) Assignee: Cold Spring Harbor Laboratory, Cold Spring Harbor, NY (US)

(21) Appl. No.: 15/546,106

(22) PCT Filed: Jan. 26, 2016
(86) PCT No.: PCT/US2016/014862

§ 371 Date: Jul. 25, 2017

Related U.S. Application Data
(60) Provisional application No. 62/107,991, filed on Jan. 26, 2015.
(60) Provisional application No. 62/108,426, filed on Jan. 27, 2015.

(51) Int. Cl. C12Q 1/68; G01N 33/68

(52) U.S. Cl. CPC C12Q 1/6876; G01N 33/6803; C12Q 1/6897; C12Q 2600/156; C12Q 2600/178

(57) ABSTRACT

Provided herein, in some aspects, are methods of determining whether a candidate protein, more specifically a functional domain of a candidate protein, is essential for viability of cells of interest using clustered regularly interspaced short palindromic repeat (CPJSPR)-Cas9 technology which holds great promise for genetic screening and for the discovery of therapeutic targets.

### RELATED APPLICATIONS

This application claims the benefit under 35 U.S.C. §119(e) of U.S. provisional application number 62/107,991, filed Jan. 26, 2015, and U.S. provisional application number 62/108,426, filed Jan. 27, 2015, each of which is incorporated by reference herein in its entirety.

### FEDERALLY SPONSORED RESEARCH

This invention was made with Government support under Grant No. CA174793, awarded by National Institutes of Health, and Grant No. CA45508, awarded by National Cancer Institute. The Government has rights in the invention.

### BACKGROUND OF INVENTION

Clustered regularly interspaced short palindromic repeat (CRISPR)-Cas9 technology holds great promise for genetic screening and for the discovery of therapeutic targets.

### SUMMARY OF INVENTION

CRISPR/Cas9 technologies exploit the ability of the Cas9 endonuclease to cleave DNA targets specified by a “single guide RNA,” or “sgRNA,” containing, for example, a 20-base match to a genomic target. Co-expressing the sgRNA with Cas9 in cells of interest can efficiently generate mutations in a target sequence. CRISPR/Cas9-mediated cleavage of a target gene results in both DNA strands being cleaved within the target sequence. Cas9 is a double-stranded DNA endonuclease that depends on interaction with the sgRNA for DNA cleavage. The resulting double-stranded break at the target site is usually repaired by the non-homologous end-joining (NHEJ) DNA repair pathway. This usually results in loss of a few, to several hundred, nucleotides around the cleavage site (referred to as a deletion mutation), although insertions are sometimes observed (referred to as an insertion mutation). Thus, when CRISPR/Cas9 is targeted to gene coding regions, it efficiently creates mutations that are often deleterious and/or effectively null alleles, however, the resulting mutations could be in-frame. The position within the gene may affect the severity of mutations in a gene-dependent manner. Thus, a variety of mutations may be generated by CRISPR/Cas9-targeting.

Typically, the sgRNA bases used for target recognition are the first 20 bases and the last 2 bases (e.g. GG). Combined, this target is sufficiently long enough that most targets of interest will turn out to be unique in mammalian genomes. Nonetheless, Cas9 can tolerate mismatches, leading to concerns about off-target cleavage. Off-target cleavage events can occur and are well documented for CRISPR/Cas9. A “seed region” of approximately 12 bases proximal to a protospacer-adjacent motif (PAM) motif is important for pairing and DNA cleavage, while mispairing in the distal bases can sometimes be tolerated. The frequency of off-target CRISPR/Cas9 cleavage events is likely target- and system-dependent.

To achieve optimal performance in negative selection screens, it is critical for CRISPR/Cas9 to generate homozygous loss-of-function mutations in a highly efficient manner, controlling for off-target cleavage events. Provided herein are CRISPR/Cas9-based strategies that, in some embodiments, exploit this principle and simultaneously reveal protein domains that support cancer maintenance. By targeting CRISPR/Cas9-mediated mutagenesis (referred to more simply as CRISPR-mediated mutagenesis) to exons encoding functional protein domains, negative selection phenotypes can be achieved that are an order of magnitude stronger than those observed through mutagenesis of, for example, 5′ exons. Also provided herein are deep sequencing-based methods for target validation that effectively exclude off-target effects. Surprisingly sequencing analyses (e.g., deep-sequencing analyses) of the present disclosure reveal that in-frame CRISPR-induced indel mutations, when they occur outside of functional protein domains, have much less of a loss-of-function phenotypic effect relative to frameshift/nonsense CRISPR-induced indel mutations that occur outside of functional protein domains. By contrast, in-frame mutations and frameshift/nonsense mutations, when they occur inside a functional protein domain, have similar loss-of-function phenotypic effect relative to each other and relative to frameshift/nonsense mutations occurring in outside of a functional domain. Thus, in-frame mutations can limit the efficacy of negative-selective CRISPR screens. This limitation can be overcome using the methods provided herein by designing sgRNAs that target functional protein domains.

The methods of the present disclosure are benchmarked by mutagenizing 34 lysine methyltransferase (KMT) domains in MLL-AF9 leukemia cells, which confirmed known cancer dependencies and identified additional disease requirements. A broad application of the methods provided herein may permit, for example, a comprehensive annotation of targetable protein domains that sustain cancer cell viability.

Some aspects of the present disclosure provide methods of determining whether a candidate protein, or more specifically, whether a functional domain of a candidate protein, is essential for viability of cells of interest. In some embodiments, the methods comprise (a) introducing, into a subpopulation of a population of Cas9-expressing cells of interest, a nucleic acid encoding a single guide RNA (sgRNA) that targets a first region of a gene (e.g., allele) encoding a candidate protein, wherein the first region encodes a functional domain of the candidate protein, thereby producing a first population of cells comprising a subpopulation of cells that comprise Cas9 nuclease and sgRNA that targets the first region, (b) introducing, into a subpopulation of a population of Cas9-expressing cells of interest, a nucleic acid encoding a sgRNA that targets a second region of a gene (e.g., allele) encoding the candidate protein, wherein the second region is 5′ to the first region and does not encode a functional domain of the candidate protein, thereby producing a second population of cells comprising subpopulation of cells that comprise Cas9 nuclease and sgRNA that targets the second region, (c) culturing the first population of cells produced in (a) and the second populations of cells produced in (b) under conditions that result in CRISPR-induced indel mutagenesis of the first region and of the second region, thereby producing a first population of cultured cells and a second population of cultured cells (e.g., the first population comprising a subpopulation of cells comprising a mutation in the first region of each gene that encodes the protein of interest, and the second population comprising a subpopulation of cells comprising a mutation in the second region of each gene that encodes the protein of interest), (d) assessing the normalized percentage of sgRNA-positive cells (NP) over time in the first population of cultured cells to determine a decrease over time in the NP for the first population of cultured cells, (e) assessing the NP over time in the second population of cultured cells to determine a decrease over time in the NP for the second population of cultured cells, and (f) comparing the decrease in NP for the first population (ΔNP1) to the decrease in NP for the second population (ΔNP2), wherein if ΔNP1 is greater than ΔNP2, the functional domain of the candidate protein is essential for viability of cells of interest. In some embodiments, the methods comprise (d) assessing a difference in the normalized percentage of sgRNA-positive cells over time in the first population of cultured cells, thereby producing a first percent difference, (e) assessing a difference in the normalized percentage of sgRNA-positive cells over time in the second population of cultured cells, thereby producing a second percent difference, and (f) comparing the first percent difference to the second percent difference, wherein if the first percent difference is a decrease that is statistically significantly greater than the second percent difference, the functional domain of the candidate protein is essential for viability of cells of interest.

Some aspects of the present disclosure provide methods of determining whether a functional domain of a candidate protein is essential for viability of cells of interest, the methods comprising (a) introducing, into a subpopulation of a population of Cas9-expressing cells of interest, a nucleic acid encoding a single guide RNA (sgRNA) that targets a first region of a gene encoding a candidate protein, wherein the first region encodes a functional domain of the candidate protein, thereby producing a first population of cells comprising a subpopulation of cells that comprise Cas9 nuclease and sgRNA that targets the first region, (b) introducing, into a subpopulation of a population of Cas9-expressing cells of interest, a nucleic acid encoding a sgRNA that targets a second region of a gene encoding the candidate protein, wherein the second region is 5′ to the first region and does not encode a functional domain of the candidate protein, thereby producing a second population of cells comprising subpopulation of cells that comprise Cas9 nuclease and sgRNA that targets the second region, (c) culturing the first population of cells produced in (a) and the second populations of cells produced in (b) under conditions that result in CRISPR-induced indel mutagenesis of the first region and of the second region, thereby producing a first population of cultured cells and a second population of cultured cells, (d) assessing the normalized percentage of CRISPR-induced indel mutations (NP) over time in the first population of cultured cells to determine a decrease over time in the NP for the first population of cultured cells, (e) assessing the NP over time in the second population of cultured cells to determine a decrease over time in the NP for the second population of cultured cells, and (f) comparing the decrease in NP for the first population (ΔNP1) to the decrease in NP for the second population (ΔNP2), wherein if ΔNP1 is greater than ΔNP2, the functional domain of the candidate protein is essential for viability of cells of interest. In some embodiments, methods comprise (d) assessing a difference in the normalized percentage of CRISPR-induced indel mutations in cells over time in the first population of cultured cells, thereby producing a first percent difference, (e) assessing a difference in the normalized percentage of CRISPR-induced indel mutations in cells over time in the second population of cultured cells, thereby producing a second percent difference, and (f) comparing the first percent difference to the second percent difference, wherein if the first percent difference is a decrease that is statistically significantly greater than the second percent difference, the functional domain of the candidate protein is essential for viability of cells of interest.

In some embodiments, methods further comprise assessing the normalized relative abundance of in-frame mutations in cells (NRA-IF) over time in the first population of cultured cells to determine a decrease over time in the NRA-IF for the first population of cultured cells, assessing the NRA-IF over time in the second population of cultured cells to determine a decrease over time in the NRA-IF for the second population of cultured cells, and comparing the decrease in NRA-IF for the first population (ΔNRA-IF1) to the decrease in NRA-IF for the second population (ΔNRA-IF2), wherein if ΔNRA1 is greater than ΔNRA-IF2, the functional domain of the candidate protein is confirmed to be essential for viability of cells of interest.

In some embodiments, methods further comprise assessing the normalized relative abundance of frameshift/nonsense mutations in cells (NRA-F/N) over time in the second population of cultured cells to determine a decrease over time in the NRA-F/N for the second population of cultured cells, assessing the normalized relative abundance of in-frame mutations in cells (NRA-IF) over time in the second population of cultured cells to determine a decrease over time in the NRA-IF for the second population of cultured cells, and comparing the decrease in NRA-F/N for the second population (ΔNRA-F/N1) to the decrease in NRA-IF for the second population (ΔNRA-IF2), wherein a ΔNRA-F/N1 that is greater than a ΔNRA-IF2 indicates limited occurrence of off-target effects resulting from CRISPR-induced indel mutagenesis.

In some embodiments, the Cas9-expressing cells of (a) and (b) further express a reporter protein (e.g., fluorescent protein such as GFP).

In some embodiments, the encoding the sgRNA of (a) and of (b) each further encode a reporter protein (e.g., fluorescent protein such as GFP).

In some embodiments, the normalized percentage of sgRNA-positive cells is assessed by assessing the normalized percentage of reporter protein-positive cells.

In some embodiments, the cells of interest are cancer cells. In some embodiments, the cells of interest are immune cells.

In some embodiments, the Cas9-expressing cells of interest of (a) and of (b) are clonal Cas9⁺ genomically-stable cells derived from the same cell line.

In some embodiments, the nucleic acid encoding the sgRNA of (a) and of (b) each is introduced through lentiviral transduction of the Cas9-expressing cells of interest. Some aspects of the present disclosure provide methods of determining whether a protein (or a functional protein domain) is essential for viability of cells of interest, comprising (a) introducing into cells of interest that express Cas9 nuclease a nucleic acid encoding a single guide RNA (sgRNA) that targets an exon encoding a functional domain of a protein, thereby producing cells that comprise Cas9 nuclease and sgRNA, (b) culturing cells produced in (a) under conditions that result in expression of a mutated exon, and (c) assessing over time, in the cultured cells of (b), the number of sgRNA-positive cells, wherein a depletion of sgRNA-positive cells by at least 2-fold (e.g., at least 3-fold, at least 5-fold, at least 10-fold, at least 15-fold, at least 20-fold, at least 50-fold) over time indicates that protein comprising the functional domain encoded of (a) is essential for viability of the cells of interest.

Some aspects of the present disclosure provide libraries of (e.g., comprising or consisting of) nucleic acids encoding sgRNAs that target functional protein domains (e.g., and do not target regions outside of function protein domains). In some embodiments, a library comprises 10 to 100,000 nucleic acids encoding sgRNAs that target functional protein domains. For example, a library may comprise 10 to 100, 10 to 1000, 10 to 10000, 100 to 1000, 100 to 10000, or 1000 to 10000 nucleic acids encoding sgRNAs that target functional protein domains.

Some aspects of the present disclosure provide compositions that include a population of Cas9-expressing cells comprising a subpopulation of cells that comprise a nucleic acid encoding a single guide RNA (sgRNA) that targets a first region of an gene encoding a candidate protein, wherein the first region encodes a functional domain of the candidate protein. Some aspects of the present disclosure provide compositions that include a population of Cas9-expressing cells comprising a subpopulation of cells that comprise a nucleic acid encoding a sgRNA that targets a second region of a gene encoding the candidate protein, wherein the second region is 5′ to the first region and does not encode a functional domain of the candidate protein.

### BRIEF DESCRIPTION OF DRAWINGS

The accompanying drawings are not intended to be drawn to scale. For purposes of clarity, not every component may be labeled in every drawing.

FIGS. 1A-1J show data collected from negative selection CRISPR experiments in MLL-AF9/Nras^{G12D} acute myeloid leukemia cells.

FIGS. 2A-2H show data demonstrating how single-guide ribodeoxynucleic acids (sgRNAs) that target Brd4 and Smarca4 functional domains lead to improved performance in negative selection experiments.

FIGS. 3A-3H show data demonstrating that a lysine methyltransferase (KMT) domain-focused CRISPR screen in MLL-AF9 leukemia validates known drug targets and reveals additional dependencies.

FIGS. 4A-4C show data obtained from a SURVEYOR assay analysis of indel mutations induced by various Brd4 or Smarca4 sgRNAs. FIG. 4A: top panel, location of Brd4 sgRNAs used in FIG. 1 relative to the domain architecture of Brd4 bromodomain; bottom panel, SURVEYOR assay of indel mutations of corresponding Brd4 genomic DNA region at day 3 post-transduction by indicated sgRNAs. sgRNA targeting ROSA26 locus serves as negative control. The GFP+/sgRNA+ percentages of each sample are labeled under the gel image. Indel frequencies were calculated by the intensity of DNA band using ImageJ software. The normalized indel % was calculated by correcting for the transduction/GFP percentage. Representative image of two independent experiments is shown. FIGS. 4C-4C: SURVEYOR assay of indel mutations of Brd4 or Smarca4 genomic DNA region induced by indicated sgRNAs at various time points post-infection. Representative image of two independent experiments is shown. M, marker.

FIGS. 5A-5H show data demonstrating validation of hits obtained from the KMT screen in RN2c. Results from a negative selection competition assay are plotted as the percentage of sgRNA/GFP⁺ cells over time following transduction of RN2c with the indicated sgRNAs. The GFP⁺ percentage is normalized to day 2 measurements. n=3. The fold-change numbers indicate GFP % (d2/d12). sgRNA targeting ROSA26 control locus is serving as a negative control. n=3. All error bars in this figure represent SEM.

FIG. 6 shows data obtained from a domain-focused KMT screen performed in Cas9⁺ NIH3T3 fibroblast cells. Negative selection is represented as the fold change of GFP⁺ cells during 22 days in culture. Each bar represents an independent sgRNA targeting the indicated KMT domain. ROSA26 is a negative control sgRNA. The x-axis was limited to a 20-fold maximum for visualization purposes.

### DESCRIPTION OF THE INVENTION

The RNA-guided endonuclease Cas9, a component of the type II CRISPR (clustered regularly interspaced short palindromic repeats) system of bacterial host defense, is a powerful tool for genome editing. Ectopic expression of Cas9 and a single guide RNA (sgRNA) is sufficient to direct the formation of double-strand breaks (DSBs) at a specific genomic region of interest. In the absence of a homology-directed repair DNA template, these DSBs become repaired in an error-prone manner through the non-homologous end joining (NHEJ) pathway to generate an assortment of short deletion and insertion mutations (collectively referred to as “indel mutations” or “indels”) in the vicinity of the sgRNA recognition site. Thus, a sgRNA designed to target a nucleic acid region of interest such as, for example, a particular exon encoding a functional domain of a protein of interest, will generate a mutation in each gene that encodes the protein of interest. This approach has been widely utilized to generate gene-specific knockouts in a variety of model systems.

Recent studies demonstrate the use of CRISPR for genetic screens in mammalian cell culture, which relied on sgRNA libraries that target constitutive 5′ coding exons to achieve gene inactivation. The capabilities of CRISPR screening are particularly evident in positive selection experiments, such as identifying mutations that confer drug resistance. In the setting of negative selection, sgRNA hits are statistically enriched for essential gene classes (e.g., ribosomal, RNA processing, and DNA replication factors), however, the overall accuracy of CRISPR for annotating genetic dependencies (for example, genes required for cell viability) is unclear. The heterogeneity of indel mutations generated using CRISPR presents a unique challenge for negative selection screens, because a loss of cell viability would be expected to require the efficient generation of homozygous loss-of-function mutations. Another technical issue with CRISPR-based screening is the occurrence of off-target mutagenesis at genomic sites with imperfect sgRNA complementarity.

The overall performance of CRISPR for genetic screening is influenced by several experimental parameters, including the level of Cas9 expression, sgRNA sequence features, off-target cutting, and the local chromatin structure near the cut-site. Results provided herein show that the performance of CRISPR in negative selection experiments is substantially improved when Cas9 cutting is directed to sequences that encode functionally important protein domains. This leads to an important principle for CRISPR screens that aim to identify cancer dependencies suitable for pharmacological inhibition, which is that sgRNA libraries may be designed to target exons that encode druggable protein domains. “Druggable” protein domains are protein domains that are amenable, or responsive, to chemical/pharmacological inhibition. This would directly link the severity of negative selection phenotypes to the functional importance of the domain being targeted. This may be particularly important for genes that encode large multi-domain proteins, but less important for small proteins, such as Rpa3. The capabilities of the methods provided herein were validated by probing a class of epigenetic targets in a genetically-engineered mouse leukemia model, although cells of interest are not limited to cancer cells. Similar observations are expected to be relevant for any CRISPR-based negative selection screen.

Domain-focused CRISPR screens provide several advantages over RNAi for studying cancer dependencies. Rapid identification of essential protein domains and the ability to rule out off-target effects can be a challenge when using RNAi, but can be readily addressed using the methodology described herein. While RNAi can be used for studying dosage effects, which is an important consideration when establishing feasibility of a target for chemical inhibition, the close correspondence between phenotypes observed using RNAi-and CRISPR-based gene perturbations throughout the studies provided herein highlights how integrating these two approaches, in some embodiments, can lead to a robust annotation of therapeutically-relevant cancer cell dependencies.

Some aspects of the present disclosure provide methods of determining whether a candidate protein, or more specifically, whether a functional domain of a candidate protein, is essential for viability of cells of interest. In some embodiments, the methods comprise (a) introducing, into a subpopulation of a population of Cas9-expressing cells of interest, a nucleic acid encoding a single guide RNA (sgRNA) that targets a first region of a gene encoding a candidate protein, wherein the first region encodes a functional domain of the candidate protein, thereby producing a first population of cells comprising a subpopulation of cells that comprise Cas9 nuclease and sgRNA that targets the first region, (b) introducing, into a subpopulation of a population of Cas9-expressing cells of interest, a nucleic acid encoding a sgRNA that targets a second region of a gene (e.g., allele) encoding the candidate protein, wherein the second region is 5′ to the first region and does not encode a functional domain of the candidate protein, thereby producing a second population of cells comprising subpopulation of cells that comprise Cas9 nuclease and sgRNA that targets the second region, (c) culturing the first population of cells produced in (a) and the second populations of cells produced in (b) under conditions that result in CRISPR-induced indel mutagenesis of the first region and of the second region, thereby producing a first population of cultured cells and a second population of cultured cells, the first population comprising a subpopulation of cells comprising a mutation in the first region of each gene that encodes the protein of interest, and the second population comprising a subpopulation of cells comprising a mutation in the second region of each gene that encodes the protein of interest, (d) assessing a difference in the normalized percentage of sgRNA-positive cells over time in the first population of cultured cells, thereby producing a first percent difference, (e) assessing a difference in the normalized percentage of sgRNA-positive cells over time in the second population of cultured cells, thereby producing a second percent difference, and (f) comparing the first percent difference to the second percent difference, wherein if the first percent difference is a decrease that is statistically significantly greater than the second percent difference, the functional domain of the candidate protein is essential for viability of cells of interest.

“Cells of interest” may be any cell type of interest. In some embodiments, cells of interest are cancer cells. For example, cancer cells of interest may be adrenal cancer cells, breast cancer cells, brain cancer cells, bone cancer cells, cervical cancer cells, colon cancer cells, endometrial cancer cells, esophageal cancer cells, gastrointestinal cancer cells, kidney cancer cells, leukemia cells, liver cancer cells, lung cancer cells, lymphoma cells, nasopharyngeal cancer cells, ocular cancer cells, oral cancer cells, ovarian cancer cells, pancreatic cancer cells, prostate cancer cells, sarcoma cells, skin cancer cells (e.g., melanoma cells), stomach cancer cells, testicular cancer cells, uterine cancer cells, and vaginal cancer cells.

In some embodiments, cells of interest are immune cells. For example, immune cells of interest may be B cells, dendritic cells, granulocytes, innate lymphoid cells, megakaryotypes, monocytes, macrophages, natural killer cells, platelets, red blood cells, T cells and thymocytes.

In some embodiments, cells of interest are stem cells (e.g., pluripotent stem cells). A “stem cell” refers to a cell with the ability to divide for indefinite periods in culture and to give rise to specialized cells. A “pluripotent stem cell” refers to a type of stem cell that is capable of differentiating into all tissues of an organism, but not alone capable of sustaining full organismal development. A “human induced pluripotent stem cell,” or “hiPS cell,” refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human iPS cells express stem cell markers and are capable of generating cells characteristic of all three germ layers (ectoderm, endoderm, mesoderm). Human iPS cells can be produced, for example, by expressing four transcription factor genes encoding OCT4, SOX2, KLF4 and c-MYC.

“Cas9-expressing cells of interest” may be any of the cells of interest described above that expresses Cas9 endonuclease. Cas9 may be expressed in the cell genomically or episomally. An example of a clonal Cas9⁺ line, which is diploid and remains genomically stable during passaging, is described in Example 1. Cas9 (CRISPR associated protein 9) is an RNA-guided DNA endonuclease enzyme associated with the CRISPR (Clustered Regularly Interspersed Palindromic Repeats) adaptive immunity system in Streptococcus pyogenes, among other bacteria. The sgRNA/Cas9 complex is recruited to a target sequence by the base-pairing between the sgRNA sequence and the complement to the target sequence in the genomic DNA. For successful binding of Cas9, the genomic target sequence should contain the correct protospacer adjacent motif (PAM) sequence immediately following the target sequence. The binding of the gRNA/Cas9 complex localizes the Cas9 to the genomic target sequence so that the wild-type Cas9 can cut both strands of DNA causing a double strand break (DSB). Cas9 will cut approximately 3-4 nucleotides upstream of the PAM sequence. Repair of a through the non-homologous end joining (NHEJ) repair pathway often results in inserts/deletions (indels) at the DSB site that can lead to frameshifts and/or premature stop codons, effectively disrupting the open reading frame (ORF) of the targeted gene.

“Transient cell expression” herein refers to expression by a cell of a nucleic acid that is not integrated into the nuclear genome of the cell. By comparison, “stable cell expression” herein refers to expression by a cell of a nucleic acid that remains in the nuclear genome of the cell and its daughter cells. Typically, to achieve stable cell expression, a cell is co-transfected with a nucleic acid encoding a marker protein (referred to as a marker gene) and an exogenous nucleic acid that is intended for stable expression in the cell (e.g., a nucleic acid encoding Cas9). The marker gene gives the cell some selectable advantage (e.g., resistance to a toxin, antibiotic, or other factor). Few transfected cells will, by chance, have integrated the exogenous nucleic acid into their genome. If a toxin, for example, is then added to the cell culture, only those few cells with a toxin-resistant marker gene integrated into their genomes will be able to proliferate, while other cells will die. After applying this selective pressure for a period of time, only the cells with a stable transfection remain and can be cultured further. In some embodiments, puromycin, an aminonucleoside antibiotic, is used as an agent for selecting stable transfection of cells of interest. Thus, in some embodiments, cells of interest are modified to express puromycin N-acetyltransferase, which confers puromycin resistance to cells of interest expressing puromycin N-acetyltransferase. Other marker genes/selection agents may be used as provided herein. Examples of such marker genes and selection agents include, without limitation, dihydrofolate reductase with methotrexate, glutamine synthetase with methionine sulphoximine, hygromycin phosphotransferase with hygromycin, and neomycin phosphotransferase with Geneticin, also known as G418.

A “population” of cells may comprise a homogenous (e.g., cells of the same type, e.g., genotype and/or phenotype) or heterogeneous (e.g., cells of different types) population of cells. In some embodiments, a population of cells comprises cells derived from the same lineage (e.g., clonal Cas9-expressing cells).

Typically, a population of cells, as provided herein, comprises at least two subpopulations of cells. For example, one subpopulation may be transfected with a nucleic acid encoding a single guide RNA (sgRNA), as provided herein, and another subpopulation may be non-transfected, or transfected with empty vector as a control. A “subpopulation” of a population of cells may comprise any number of cells from a particular cell population. In some embodiments, a subpopulation includes z5% to 95% of a population. For example, a subpopulation may include 5%, 10%, 15%, 20%, 25%, 30%, 35%, 40%, 45%, 50%, 55%, 60%, 65%, 70%, 75%, 80%, 85%, 90% or 95% of a population.

Herein, a “first” population of cells and a “second” populations of cells typically refer to separate physically-separate populations (e.g., separate cell cultures in separate culture flasks/wells/dishes), although each may be derived, e.g., clonally, from the same cell line. In some embodiments, “first” and “second” populations are manipulated in parallel, as described herein. For example, a first population may be transfected with a nucleic acid encoding a sgRNA that targets a first region of a gene encoding a functional protein domain, while in parallel, or sequentially (close in time), a second population may be transfected with a nucleic acid encoding a sgRNA that targets a second region of a gene located upstream of the first region.

A “candidate protein” refers to any protein of interest that may function in cell maintenance (e.g., cell viability). For example, a candidate protein may function in cell cycle progression, replication, differentiation or apoptosis. In some embodiments, a candidate protein (and/or a candidate protein domain) is a cancer drug target. In some embodiments, a candidate protein (and/or a candidate protein domain) is a small molecule drug target. In some embodiments, a candidate protein (and/or a candidate protein domain) is responsive or amenable to chemical or pharmacological inhibition.

Non-limiting examples of candidate proteins include G protein couple receptor family proteins, kinase (e.g., tyrosine, serine/threonine kinase, e.g., based on the kinome list from Manning et al. Science 2002, incorporated by reference), enzymes with catalytic function (e.g., acetlytransferase, methyl transferase, demethylase, de-acetlytransferase), proteases, phosphatases, proteins having an ATPase domain, proteins having a post-translation modification reader domain, (e.g., bromodomain, PHD domain, chromodomain), ion channel proteins and nuclear receptors. Other candidate proteins may be used as provided herein.

A “functional domain of a candidate protein” refers to a conserved part of a given protein sequence and (e.g., tertiary) structure that can function and exist independently of the rest of the protein chain. Conserved domains of a candidate protein can be identified using, for example, the National Center for Biotechnology Information (NCBI) website: in particular, the conserved domain annotation under the “refSeq section” of the gene information may be used. Other means of identifying/selecting candidate proteins are known in the art and contemplated herein (see, e.g., dgidb.genome.wustl.edu/downloads/walkthroughUpdated.pdf; and ebi.ac.uk/chembl/drugebility/faq).

A functional domain of a candidate protein, also referred to as a “functional protein domain,” is considered “essential” for cell viability if a deleterious mutation in that domain—e.g., in both genes/alleles encoding the protein containing that domain—causes death of the cell over time (e.g., 1 to 10 days, e.g., 1, 2, 3, 4, 5, 6, 7, 8, 9 or 10 days, or more).

A “nucleic acid” refers to at least two nucleotides covalently linked together, and in some instances, may contain phosphodiester bonds (e.g., a phosphodiester “backbone”). The nucleic acid may be DNA, both genomic and cDNA, RNA or a hybrid, where the nucleic acid contains any combination of deoxyribo-and ribonucleotides, and any combination of bases, including uracil, adenine, thymine, cytosine, guanine, inosine, xathanine hypoxathanine, isocytosine, and isoguanine. The nucleic acids may be single-stranded (ss) or double-stranded (ds), as specified, or may contain portions of both single-stranded and double-stranded sequence. Nucleic acids, as provided herein, may be naturally occurring, recombinant or synthetic. “Recombinant nucleic acids” are molecules that are constructed by joining nucleic acid molecules and, in some embodiments, can replicate in a living cell. “Synthetic nucleic acids” are molecules that are chemically or by other means synthesized or amplified, including those that are chemically or otherwise modified but can base pair with naturally occurring nucleic acid molecules. Recombinant and synthetic nucleic acids also include those molecules that result from the replication of either of the foregoing.

As provided herein, nucleic acids encoding a single guide RNAs (sgRNAs) are introduced into cells of interest. It should be understood that a “nucleic acid encoding a sgRNA” contains the necessary genetic elements for cellular expression of the sgRNA. For example, such a nucleic acid comprises a promoter sequence (referred to simply as a “promoter”) operably linked to a nucleotide sequence encoding the sgRNA. A “promoter” refers to a control region of a nucleic acid sequence at which initiation and rate of transcription of the remainder of a nucleic acid sequence are controlled. A promoter may also contain subregions at which regulatory proteins and molecules may bind, such as RNA polymerase and other transcription factors. Promoters may be constitutive, inducible, activatable, repressible, tissue-specific or any combination thereof. An “inducible promoter” is one that is characterized by initiating or enhancing transcriptional activity when in the presence of, influenced by or contacted by an inducer or inducing agent. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence. Nucleic acids may contain additional genetic elements such as, for example, enhancers and terminators.

Nucleic acids may be introduced into cells by transformation, transfection, transduction or electroporation. Other means of introducing nucleic acids are known in the art and may be used as provided herein.

A nucleic acid encoding a sgRNA, in some embodiments, is “linked” to a nucleic acid encoding a reporter protein. A “reporter protein” refers to a protein that can be used to measure nucleic acid expression (e.g., sgRNA expression) and generally produce a reporter signal such as fluorescence, luminescence or color. The presence of a reporter protein in a cell or organism is readily observed. For example, fluorescent proteins (e.g., green fluorescent protein (GFP)) cause a cell to fluoresce when excited with light of a particular wavelength, luciferases cause a cell to catalyze a reaction that produces light, and enzymes such as β-galactosidase convert a substrate to a colored product. Reporter proteins for use as provided herein include any reporter protein described herein or known to one of ordinary skill in the art.

There are several different ways to measure or quantify a reporter protein depending on the particular reporter protein and what kind of characterization data is desired. In some embodiments, microscopy may be a useful technique for obtaining both spatial and temporal information on reporter activity, particularly at the single cell level. In some embodiments, flow cytometers can be used for measuring the distribution in reporter activity across a large population of cells. In some embodiments, plate readers may be used for taking population average measurements of many different samples over time. In some embodiments, instruments that combine such various functions may be used, such as multiplex plate readers designed for flow cytometers, and combination microscopy and flow cytometric instruments.

Fluorescent proteins may be used for visualizing or quantifying sgRNA expression. Fluorescence can be readily quantified using a microscope, plate reader or flow cytometer equipped to excite the fluorescent protein with the appropriate wavelength of light. Several different fluorescent proteins are available, thus multiple gene expression measurements can be made in parallel. Examples of genes encoding fluorescent proteins that may be used in accordance with the invention include, without limitation, those proteins provided in U.S. Patent Application No. 2012/0003630 (see Table 59), incorporated herein by reference.

Luciferases may also be used for visualizing or quantifying sgRNA expression, particularly for measuring low levels of sgRNA expression, as cells tend to have little to no background luminescence in the absence of a luciferase. Luminescence can be readily quantified using a plate reader or luminescence counter. Examples of genes encoding luciferases for that may be used in accordance with the invention include, without limitation, dmMyD88-linker-Rluc, dmMyD88-linker-Rluc-linker-PEST191, and firefly luciferase (from Photinus pyralis).

Enzymes that produce colored substrates (“colorimetric enzymes”) may also be used for visualizing or quantifying sgRNA expression. Enzymatic products may be quantified using spectrophotometers or other instruments that can take absorbance measurements including plate readers. Like luciferases, enzymes such as β-galactosidase can be used for measuring low levels of gene expression because they tend to amplify low signals. Examples of genes encoding colorimetric enzymes that may be used in accordance with the invention include, without limitation, lacZ alpha fragment, lacZ (encoding beta-galactosidase, full-length), and xylE.

The term “multiplicity of infection” or “MOI” refers to the ratio of agents (e.g., nucleic acids encoding sgRNA) to targets (e.g., Cas9-expressing cells). For example, when referring to a group of targets cells transfected with recombinant nucleic acids, the MOI is the ratio of the number of recombinant nucleic acids to the number of target cells in a defined space (e.g., a well or Petri dish). In some embodiments, a nucleic acid encoding a sgRNA is introduced into Cas9-expressing cells at a MOI of 0.2 to 9.0. For example, a nucleic acid encoding a sgRNA may be introduced into Cas9-expressing cells at a MOI of 0.2, 0.3, 0.4, 0.5, 0.6, 0.7, 0.8 or 0.9. In some embodiments, a nucleic acid encoding a sgRNA is introduced into Cas9-expressing cells at a MOI of 0.3 to 0.5.

A “CRISPR-induced indel mutation” is a class of mutations that includes insertions, deletions or combination of insertions and deletions introduced in a nucleic acid through a CRISPR-mediated mechanism, also referred to as “CRISPR-induced indel mutagenesis.” Along with Cas9 endonuclease, CRISPR experiments require the introduction of a sgRNA containing an approximately 15 to 30 base sequence specific to a target nucleic acid (e.g., DNA). sgRNA can be delivered as RNA or by transfection with a vector (e.g., plasmid) having an sgRNA-coding sequence operably linked to a promoter. In some embodiments, a sgRNA has a length of 15, 16, 17, 18, 19, 20, 21, 22, 23, 24, 25, 26, 27, 28, 29 or 30 nucleotides.

In some embodiments, a nucleic acid encoding a sgRNA is designed to target a “first region” of a gene encoding a candidate protein, wherein the first region encodes a functional domain of the candidate protein. Thus, a “first region” is typically located in a coding exon of a gene encoding a candidate protein. In some embodiments, a nucleic acid encoding a sgRNA is designed to target a “second region” of a gene encoding a candidate protein, wherein the second region is 5′ to the first region and does not encode a functional domain of the candidate protein. Thus, a “second region” is typically located “outside of a coding exon of a gene encoding a candidate protein. The term “5′,” also referred to as “upstream,” refers to a relative position in a nucleic acid. Each nucleic acid strand has a 5′” (e.g., 5′-phosphate) end and a 3′ (e.g., 3′-hydroxyl) end, so named for the carbons on the deoxyribose (or ribose) ring. By convention, upstream and downstream relate to the 5′ to 3′ direction in which RNA transcription takes place. Upstream is toward the 5′ end of the RNA molecule and downstream is toward the 3′ end. When considering double-stranded DNA, upstream is toward the 5′ end of the coding strand for the gene of interest and downstream is toward the 3′ end. Due to the anti-parallel nature of DNA, this means the 3′ end of the template strand is upstream of the gene and the 5′ end is downstream.

A sgRNA is designed to be “complementary” to a region of a gene encoding a candidate protein. Two nucleic acids are “complementary” to one another if they base-pair, or bind, to each other to form a double-stranded nucleic acid molecule via Watson-Crick interactions (also referred to as hybridization). Typically, sgRNAs are designed to be perfectly complementary (100% complementary) to a target.

Some aspects of the present disclosure comprise assessing a difference in the normalized percentage of sgRNA-positive cells over time in a given population of cultured cells. This can be achieved, for example, by culturing a population of cells for a set period of time (e.g., 10 days) and at select time points during that set period of time (e.g., day 3, day 7 and day 10) assessing the percentage of cells that express sgRNA. In instances where the nucleic acid encoding the sgRNA is linked to a reporter molecule (e.g., GFP), the percentage of cells that express sgRNA may be determined by assessing the percentage of cells that express the reporter molecule.

Cells of interest may be cultured for 1 day to 14 days, or more. In some embodiments, cells are cultures for 1 day to 3 days, 1 day to 7 days, or 1 day to 10 days. In some embodiments, cells are cultured for 1, 2, 3, 4, 5, 6, 7, 8, 9, 10, 11, 12, 13 or 14 days. In some embodiments, the percentage of cells that express sgRNA is assessed every other day, every three days, or randomly during a particular time period.

Some aspects of the present disclosure relate to the assessment of a the normalized percentage of sgRNA-positive cells (NP) over time in a first population of cultured cells to determine a decrease over time in the NP for the first population of cultured cells, assessing the NP over time in a second population of cultured cells to determine a decrease over time in the NP for the second population of cultured cells, and comparing the decrease in NP for the first population (ΔNP1) to the decrease in NP for the second population (ΔNP2), wherein if ΔNP1 is greater than ΔNP2, the functional domain of the candidate protein is essential for viability of cells of interest. In some embodiments, the ΔNP1 is at least 50% greater than the ΔNP2. For example, the ΔNP1 may be (or may be at least) 50%, 60%, 65%, 70%, 75%, 80%, 85%, 90%, 95%, 100%, 150%, 200%, 250%, 300%, 350%, 400%, 450%, 500%, 550%, 600%, 650%, 700%, 750%, 800%, 850%, 900%, 950%, 1000%, 2000%, 3000, 4000% or 5000% greater than the ΔNP2. In some embodiments, the ΔNP1 is at least 2-fold greater than the ΔNP2. For example, the ΔNP1 may be (or may be at least) 2-fold, 3-fold, 4-fold, 5-fold, 6-fold, 7-fold, 9-fold, 10-fold, 15-fold, 10-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, 1000-fold, 2000-fold, 3000-fold, 4000-fold or 5000-fold greater than the ΔNP2.

Thus, some aspects of the present disclosure provide methods of determining whether a functional domain of a candidate protein is essential for viability of cells of interest, the methods comprising (a) introducing, into a population of Cas9-expressing cells of interest, a nucleic acid encoding a single guide RNA (sgRNA) that targets a first region of an gene encoding a candidate protein, wherein the first region encodes a functional domain of the candidate protein, thereby producing a first population of cells comprising a subpopulation of cells that comprise Cas9 nuclease and sgRNA that targets the first region, (b) introducing, into a population of Cas9-expressing cells of interest, a nucleic acid encoding a sgRNA that targets a second region of a gene encoding the candidate protein, wherein the second region is 5′ to the first region and does not encode a functional domain of the candidate protein, thereby producing a second population of cells comprising subpopulation of cells that comprise Cas9 nuclease and sgRNA that targets the second region, (c) culturing the first population of cells produced in (a) and the second populations of cells produced in (b) under conditions that result in CRISPR-induced indel mutagenesis of the first region and of the second region, thereby producing a first population of cultured cells and a second population of cultured cells, (c) assessing the normalized relative abundance of in-frame mutations in cells (NRA-IF) over time in the first population of cultured cells to determine a decrease over time in the NRA-IF for the first population of cultured cells, (d) assessing the NRA-IF over time in the second population of cultured cells to determine a decrease over time in the NRA-IF for the second population of cultured cells, and (e) comparing the decrease in NRA-IF for the first population (ΔNRA-IF1) to the decrease in NRA-IF for the second population (ΔNRA-IF2), wherein if ΔNRA1 is greater than ΔNRA-IF2, the functional domain of the candidate protein is confirmed to be essential for viability of cells of interest.

Thus, some aspects of the present disclosure provide methods of determining whether a functional domain of a candidate protein is essential for viability of cells of interest, the methods comprising (a) introducing, into a population of Cas9-expressing cells of interest, a nucleic acid encoding a single guide RNA (sgRNA) that targets a first region of an gene encoding a candidate protein, wherein the first region encodes a functional domain of the candidate protein, thereby producing a first population of cells comprising a subpopulation of cells that comprise Cas9 nuclease and sgRNA that targets the first region, (b) introducing, into a population of Cas9-expressing cells of interest, a nucleic acid encoding a sgRNA that targets a second region of a gene encoding the candidate protein, wherein the second region is 5′ to the first region and does not encode a functional domain of the candidate protein, thereby producing a second population of cells comprising subpopulation of cells that comprise Cas9 nuclease and sgRNA that targets the second region, (c) culturing the first population of cells produced in (a) and the second populations of cells produced in (b) under conditions that result in CRISPR-induced indel mutagenesis of the first region and of the second region, thereby producing a first population of cultured cells and a second population of cultured cells, (d) assessing the normalized relative abundance of in-frame mutations in cells (NRA-IF) over time in the first population of cultured cells to determine a decrease over time in the NRA-IF for the first population of cultured cells, (e) assessing the NRA-IF over time in the second population of cultured cells to determine a decrease over time in the NRA-IF for the second population of cultured cells, and (f) comparing the decrease in NRA-IF for the first population (ΔNRA-IF1) to the decrease in NRA-IF for the second population (ΔNRA-IF2), wherein if ΔNRA1 is greater than ΔNRA-IF2, the functional domain of the candidate protein is confirmed to be essential for viability of cells of interest.

In some embodiments, the present disclosure provides methods of determining whether a functional domain of a candidate protein is essential for viability of cells of interest, the methods comprising (a) introducing, into a population of Cas9-expressing cells of interest, a nucleic acid encoding a single guide RNA (sgRNA) that targets a first region of an gene encoding a candidate protein, wherein the first region encodes a functional domain of the candidate protein, thereby producing a first population of cells comprising a subpopulation of cells that comprise Cas9 nuclease and sgRNA that targets the first region, (b) introducing, into a population of Cas9-expressing cells of interest, a nucleic acid encoding a sgRNA that targets a second region of a gene encoding the candidate protein, wherein the second region is 5′ to the first region and does not encode a functional domain of the candidate protein, thereby producing a second population of cells comprising subpopulation of cells that comprise Cas9 nuclease and sgRNA that targets the second region, (c) culturing the first population of cells produced in (a) and the second populations of cells produced in (b) under conditions that result in CRISPR-induced indel mutagenesis of the first region and of the second region, thereby producing a first population of cultured cells and a second population of cultured cells, (d) assessing the normalized relative abundance of frameshift/nonsense mutations in cells (NRA-F/N) over time in the second population of cultured cells to determine a decrease over time in the NRA-F/N for the second population of cultured cells, (e) assessing the normalized relative abundance of in-frame mutations in cells (NRA-IF) over time in the second population of cultured cells to determine a decrease over time in the NRA-IF for the second population of cultured cells, and (f) comparing the decrease in NRA-F/N for the second population (ΔNRA-F/N1) to the decrease in NRA-IF for the second population (ΔNRA-IF2), wherein a ANRA-F/N1 that is greater than a ΔNRA-IF2 indicates limited occurrence of off-target effects resulting from CRISPR-induced indel mutagenesis.

In some embodiments, a functional domain of a candidate protein is considered essential for viability of cells of interest if ΔNP1 is statistically significantly greater than ΔNP2. In some embodiments, a ΔNP1 that is greater than ΔNP2 is considered statistically significantly greater if it is associated with a p-value of less than (<) 0.05. In some embodiments, a ΔNP1 that is greater than ΔNP2 is considered statistically significant if it is associated with a p-value of <0.01.

“Normalized abundance” of a tracked mutation is the ratio of the number of observed mutant sequences divided by the number of wild-type sequences, normalized by the value of this same quantity on the initial day of analysis (e.g., day 3, as described in Example 1).

### EXAMPLES

### Example 1

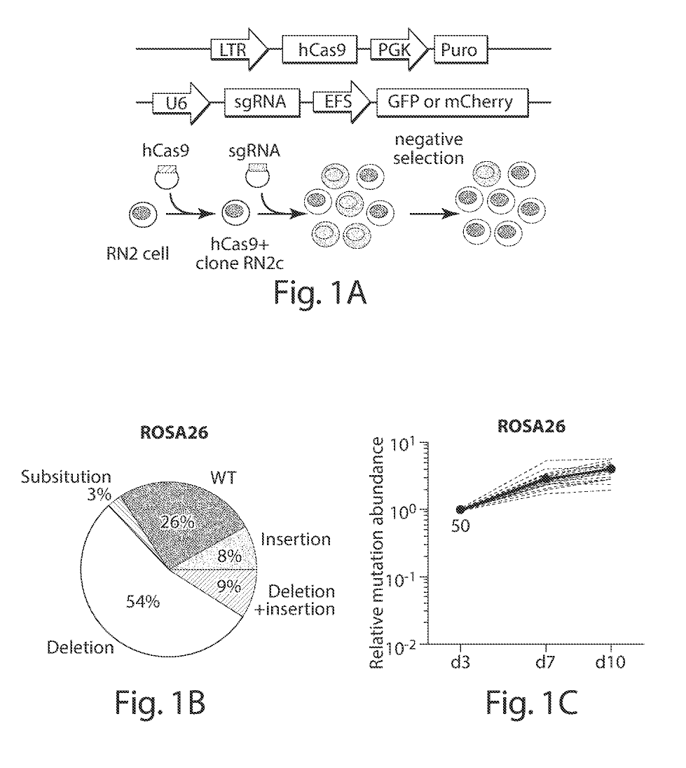

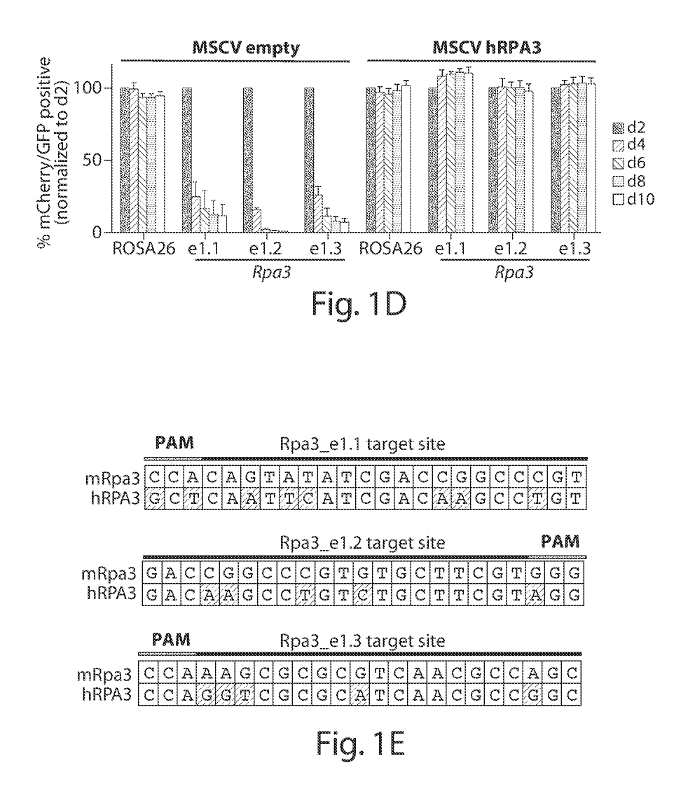

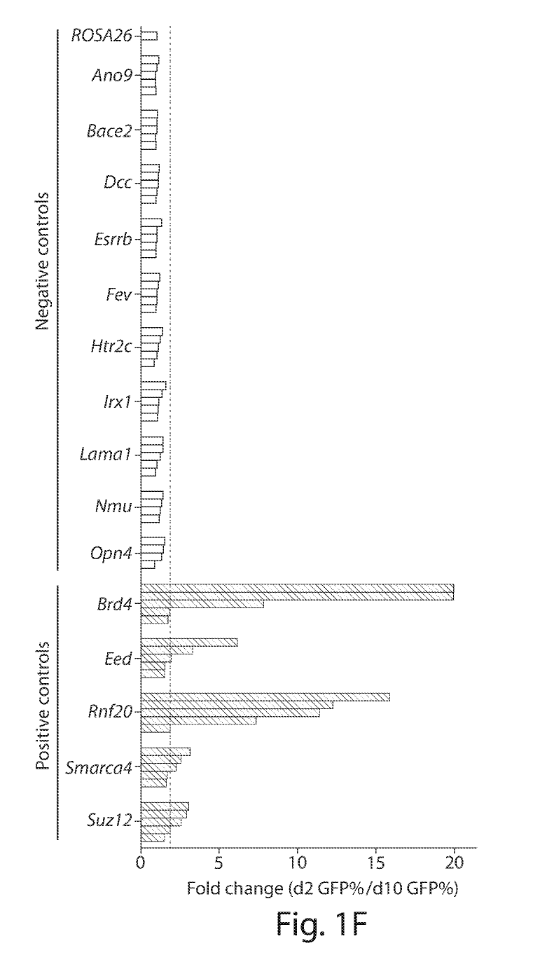

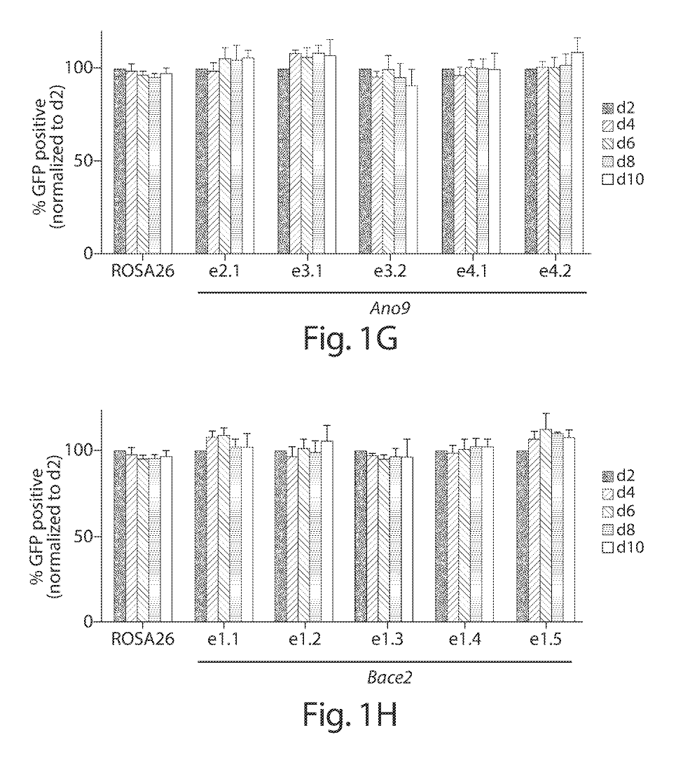

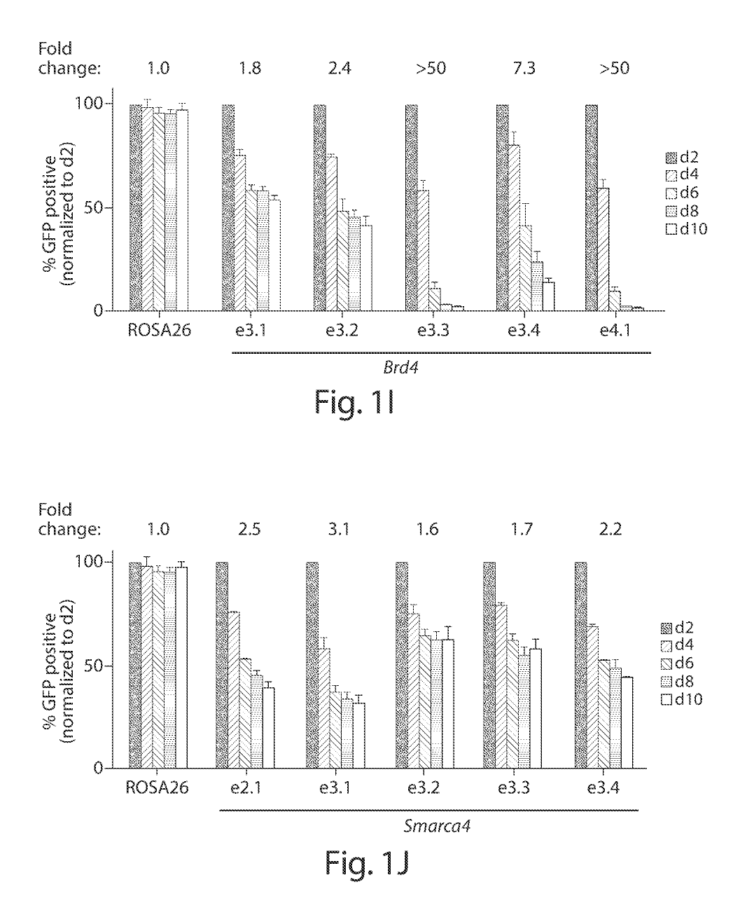

CRISPR-based mutagenesis methods provided herein are based, in part, on negative selection experiments using a murine MLL-AF9/Nras^{G12D} acute myeloid leukemia line (RN2), which has been used extensively to identify dependencies (e.g., genes essential for cell viability) using RNA interference. A clonal Cas9⁺ line (RN2c), which is diploid and remains genomically stable during passaging, was derived (FIG. 1A). Lentiviral transduction of RN2c cells with a vector expressing a GFP-linked sgRNA targeting the ROSA26 locus resulted in a high efficiency of indel mutations near the predicted cut site, which reached >95% editing efficiency by day 10 post-infection (FIG. 1B, C).

Next, how mutagenesis of an essential gene influences the maintenance of sgRNA positivity during cell culturing was examined using three sgRNAs designed to target the first exon of Rpa3, which encodes a 17 kD protein required for DNA replication. Unlike the effects of targeting ROSA26, cells expressing Rpa3 sgRNAs were rapidly outcompeted by non-transduced cells over 8 days in culture (FIG. 1C). Importantly, these effects were rescued by the presence of a human RPA3 cDNA, which has several mismatches with the mouse Rpa3 sgRNAs (FIGS. 1D, 1E). This indicates that negative selection induced by CRISPR can be attributed to mutational effects at a single target gene.

To further evaluate the performance of CRISPR mutagenesis as a negative selection screening strategy, ten additional negative control genes (chosen based on having undetectable expression in RN2) and five essential genes encoding chromatin regulators (Brd4, Smarca4, Eed, Suz12, and Rnf20) were targeted. The genes were previously identified as dependencies using shRNA-based knockdown. Four to five sgRNAs were designed to target 5′ exons of each gene, a design principle used in previous CRISPR screens. Notably, all 49 sgRNAs targeting non-expressed genes failed to undergo negative selection, suggesting a low frequency of false-positive phenotypes conferred by off-target DNA cleavage (FIGS. 1F-1H). In contrast, a large fraction of the positive control sgRNAs led to depletion of GFP-positivity, with a subset exhibiting robust depletion that exceeded 10-fold (FIG. 1F, 1I, 1J). A criterion of two or more sgRNAs depleting >2-fold accurately discriminates all of the positive controls from the negative control genes. Hence, these experiments support the capabilities CRISPR-based mutagenesis for conducting negative selection screens.

FIG. 1A-1J show data collected from negative selection CRISPR experiments in MLL-AF9/Nras^{G12D} acute myeloid leukemia cells. FIG. 1A: Experimental strategy. (top) Vectors used to derive clonal MLL-AF9; Nras^{G12D} leukemia RN2c cells that express a human codon-optimized Cas9 (hCas9) and for sgRNA transduction. GFP or mCherry reporters were used where indicated to track sgRNA negative selection. LTR: long terminal repeat promoter, PGK: phosphoglycerate kinase 1 promoter, Puro: puromycin resistance gene, U6: a Pol III-driven promoter, sgRNA: chimeric single guide RNA, EFS: EF1a promoter, GFP: green fluorescent protein. FIG. 1B: Analysis of CRISPR editing efficiency at ROSA26 locus in RN2c cells. This analysis was performed on PCR-amplified genomic regions corresponding to the sgRNA cut site. Pie chart depicts sequence variants at the ROSA26 sgRNA target site at day 10post-infection. The presence of wild-type sequence at 26% reflects the 71% GFP/sgRNA-positivity in this experiment. WT: wild-type. FIG. 1C: Relative abundance of 50 individual ROSA26 indels (indicated as light-gray lines) at indicated time points normalized to the abundance at day 3. The solid black line represents the median normalized abundance of all 50 mutations. The normalized abundance of each tracked mutation was defined as the ratio of the number of observed mutant sequences divided by the number of wild-type sequences, normalized by the value of this same quantity at day 3. FIG. 1D: Negative selection competition assay that plots the percentage of sgRNA/mCherry+ cells over time following transduction of RN2c with indicated sgRNAs. Experiments were performed in either RN2c cells transduced with an empty murine stem cell virus (MSCV) vector or MSCV expressing human RPA3 linked with a GFP reporter. The mCherry/GFP double positive percentage is normalized to day 2 measurements. el labeling of sgRNAs refers to targeting of exon 1. n=3. FIG. 1E: Comparison of mouse Rpa3 and human RPA3 sequences at the indicated sgRNA recognition sites. Location of protospacer adjacent motif (PAM) is indicated. Red color indicates mismatches. FIG. 1F: Summary of negative selection experiments with sgRNAs targeting the indicated genes. Negative selection is plotted as the fold change of GFP-positivity (d2/d10) during 8 days in culture. Each bar represents an independent sgRNA targeting a 5′ exon of the indicated gene. The dashed-line indicates a two-fold change. The fold change for two Brd4 sgRNAs was >50-fold, but the axis was limited to 20-fold maximum for visualization purposes. The data shown are the mean value of 3 independent replicates. FIGS. 1G-1J: Negative selection time-course experiments, as described in FIG. 1D. The fold-change numbers indicate GFP % (d2/d10). n=3. All error bars in this figure represent SEM.

### Example 2

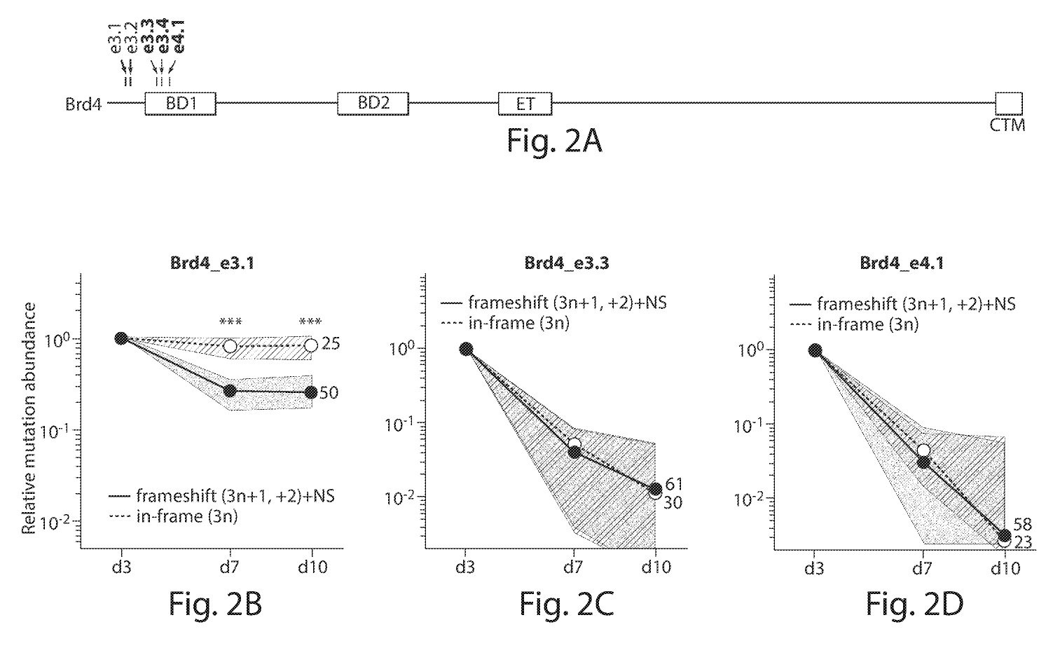

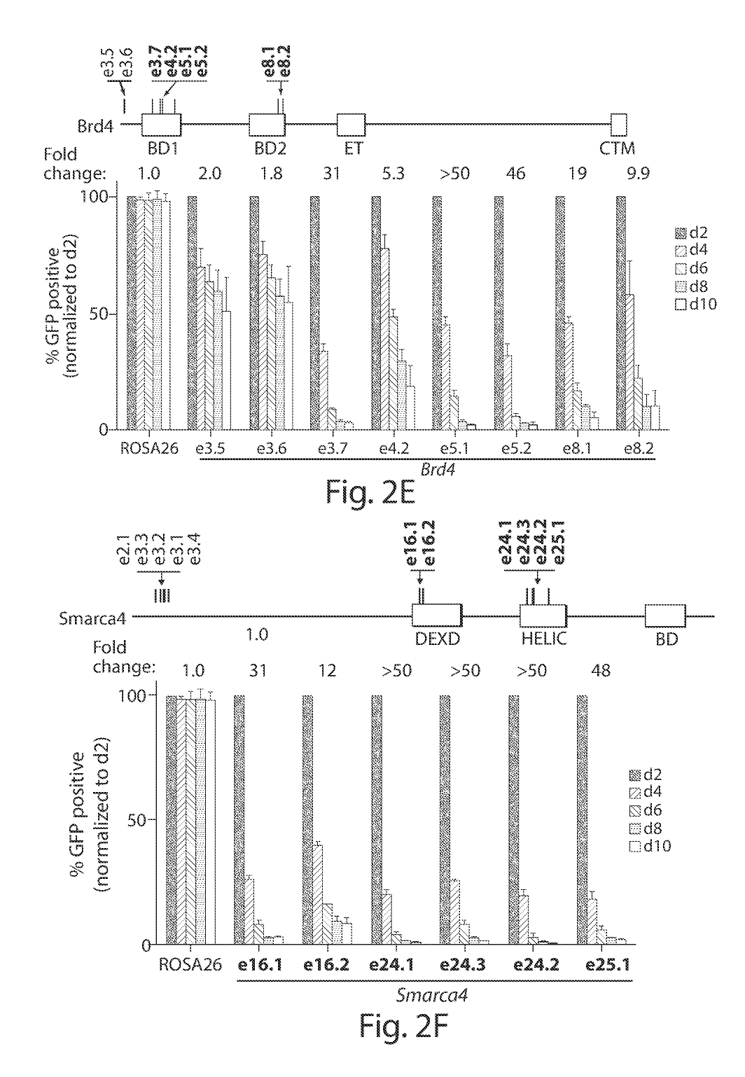

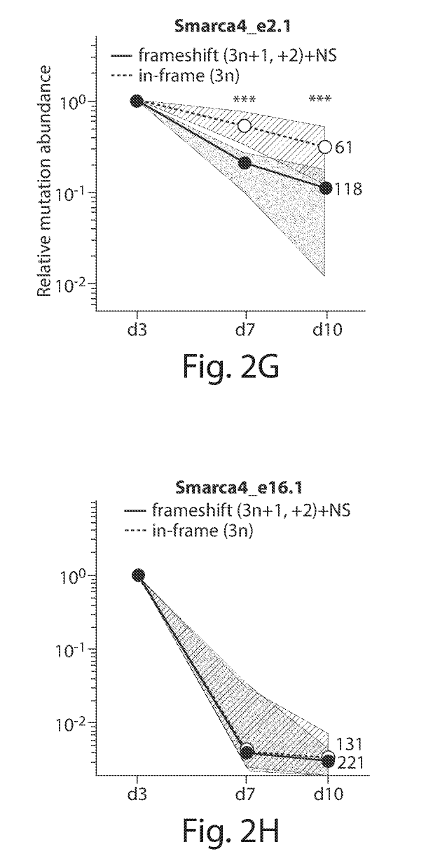

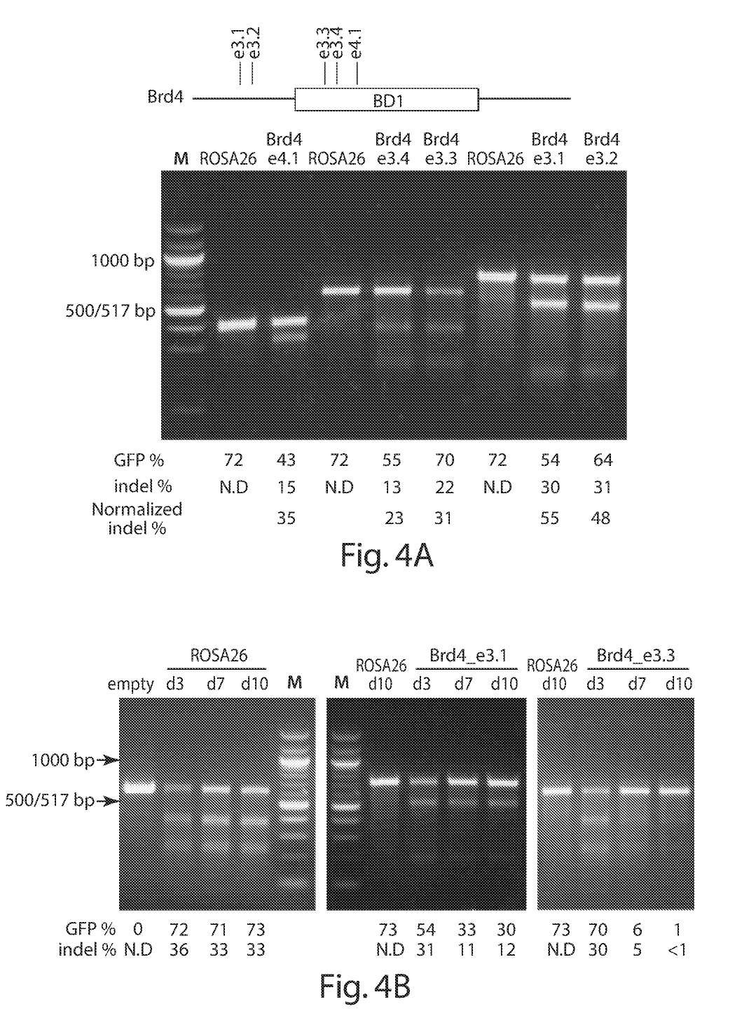

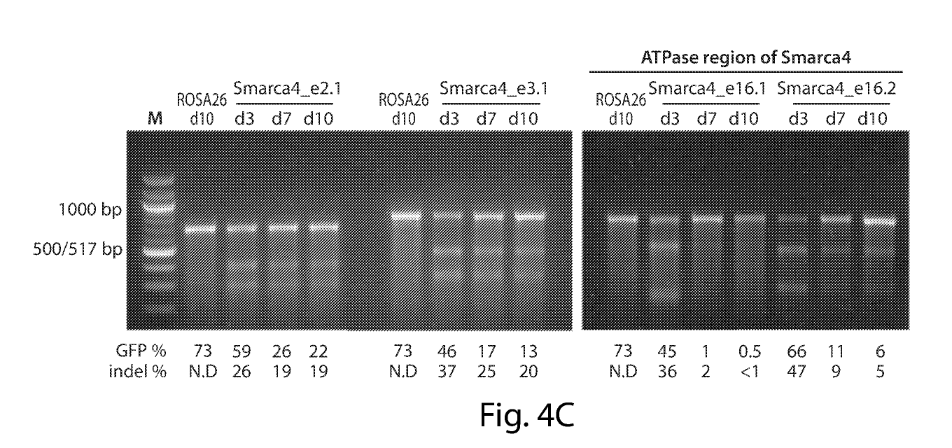

In the experiments described in Example 1, there was significant variability in the performance of individual sgRNAs targeting the same gene. For example, two of the Brd4 sgRNAs became depleted >50 fold while two were only depleted ˜2-fold over eight days in culture (FIG. 1I). Using SURVEYOR assays, data showed that the variation in phenotype severity was not due to differences in overall mutagenesis efficiency, but rather was due to stronger negative selection pressure against cells harboring the different sgRNA-induced mutations (FIGS. 4A and 4B). Interestingly, the Brd4 sgRNAs causing severe phenotypes targeted sequences encoding bromodomain 1 (BD1), while the sgRNAs causing weaker phenotypes targeted more N-terminal regions outside of the bromodomain (FIG. 2A). Prior studies showed that the bromodomains of Brd4 are necessary for leukemia maintenance, as evidenced by the anti-leukemia activity of small-molecule Brd4 bromodomain inhibitors. Without being bound by theory, it was thought that CRISPR targeting of the Brd4 BD1 region resulted in a higher percentage of deleterious mutations than CRISPR targeting of Brd4 regions outside of this critical domain.

This hypothesis was evaluated by deep sequencing of the mutagenized Brd4 exons (PCR-amplified from genomic DNA) during a negative selection time-course, which is a means to track how individual mutations impair cellular fitness. For these experiments, BD1 mutations (introduced by sgRNAs e3.3 and e4.1) were directly compared with mutations introduced outside of BD1 by sgRNA e3.1 (FIGS. 2B-2D). All three sgRNAs generated a significant number of frameshift and nonsense mutations near the predicted cut site, which, as expected, underwent negative selection when introduced at any of the three Brd4 locations (FIGS. 2B-2D). In contrast, negative selection of in-frame mutations was highly dependent on the region being targeted. The in-frame mutations generated in BD1 were negatively selected to an extent comparable to frameshift mutations (FIGS. 2C and 2D), whereas in-frame mutations occurring outside of BD1 exhibited no apparent functional impairment (FIG. 2B). Because in-frame variants represent a significant fraction of the total mutations generated by CRISPR, a BD1 sgRNA would be expected to have a higher probability of generating biallelic loss-of-function mutations than a sgRNA targeting outside of this domain. These results suggest that the variable performance of Brd4 sgRNAs in negative selection experiments is largely due to the varying functionality of in-frame mutations generated at the different cut sites, which is attributed to the functional significance of the specific protein region being targeted.

Deep sequencing-based measurement of mutation abundance provided a useful means of excluding off-target effects, which has been a confounding variable in negative selection RNAi screens. As described above, mutations induced by Brd4 sgRNA e3.1 exhibit a categorical separation of gene/allele fitness for the in-frame (functional) and frameshift/nonsense (non-functional) mutation classes (FIG. 2B). The consistency of this pattern across 75 distinct mutations provides strong evidence that the Brd4 open reading frame encodes an essential protein in leukemia cells, because this pattern would not occur if negative selection was attributed due to mutagenesis of an off-target site. Hence, this deep sequencing analysis of gene.allele functionality can be used to rigorously validate genetic dependencies.

To further strengthen the correlation between the severity of negative selection and the location of mutagenesis along the encoded target protein, additional sgRNAs targeting different regions of Brd4 were evaluated, and data show that bromodomain (BD1 or BD2) mutagenesis consistently out-performed other sites of targeting (FIG. 2E). In a prior study, it was shown that Smarca4, which encodes the Brg1 subunit of SWI/SNF complexes, requires its ATPase activity to support leukemia viability. Therefore, additional sgRNAs were designed to target the ATPase (DEXD/HELIC) domain-encoding exons of Smarca4. Remarkably, all six sgRNAs targeting this region exhibited severe phenotypes, with a GFP depletion ranging from 10-to 50-fold (FIG. 2F), whereas sgRNAs targeting 5′ exons of Smarca4 only led to ˜2-fold changes (FIG. 1J). SURVEYOR analysis also confirmed that indels occurring in the Smarca4 ATPase domain exhibited stronger negative selection than indels introduced at 5′ exons (FIG. 4C). Deep sequencing-based analysis of mutation functionality validated Smarca4 as an on-target dependency (FIG. 2G) and validated the functional significance of its ATPase domain (FIG. 2H). These results lend further support that the performance of negative selection CRISPR experiments is improved when sgRNAs are designed to target sequences that encode functional protein domains.

FIGS. 2A-2H shows data demonstrating that sgRNAs that target Brd4 and Smarca4 functional domains lead to improved performance in negative selection experiments. FIG. 2A: Location of Brd4 sgRNAs used in FIG. 1 relative to the domain architecture of Brd4 protein. BD1: bromodomain 1, BD2: bromodomain 2, ET: extra-terminal domain, CTM: C-terminal motif. (b-d) Deep sequencing analysis of mutation abundance following CRISPR-targeting of different Brd4 regions. This analysis was performed on PCR-amplified genomic regions corresponding to the sgRNA cut site at the indicated timepoints. Indel mutations were categorized into two groups: in-frame (3n) or frameshift (3n+1, 3n+2)+ nonsense (NS). Green and red numbers indicate the number of in-frame and frameshift+NS mutants that were tracked, respectively. Dots of the same color indicate the median normalized abundance at the indicated time point for all mutations within each group; shaded regions indicate the interquartile range of normalized abundance values. Significant differences between the enrichment values of the in-frame and frameshift+NS mutations were assessed using a Mann-Whitney-Wilcoxon test; ** indicates p<0.01, and *** indicates p<0.005. The normalized abundance of each tracked mutation was defined as the ratio of the number of observed mutant sequences divided by the number of wild-type sequences, normalized by the value of this same quantity at day 3. FIGS. 2G and 2H: Deep sequencing analysis of mutagenized Smarca4 exons induced by the indicated sgRNAs, as performed in FIGS. 2B-2D. All error bars in this figure represent SEM.

### Example 3

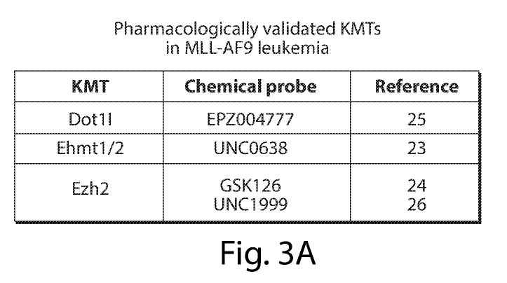

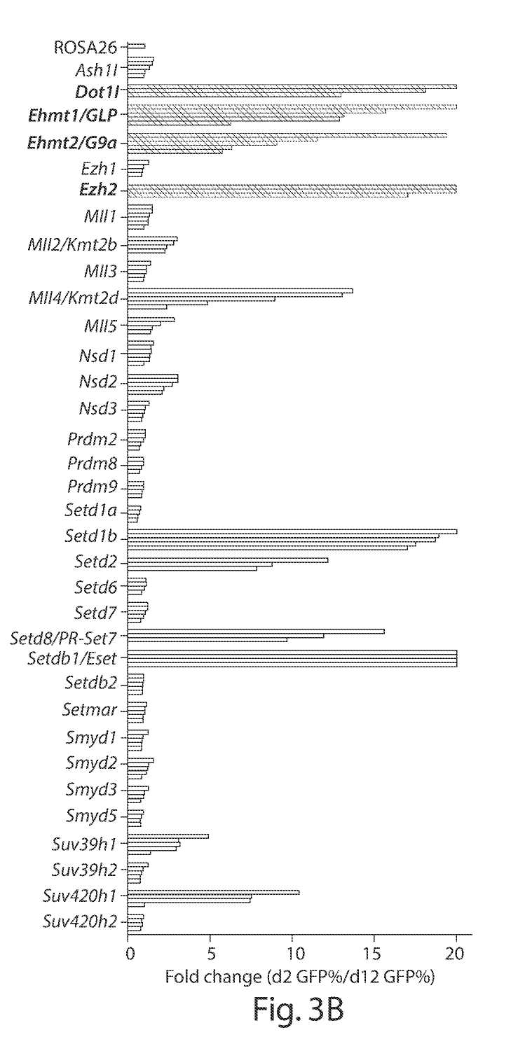

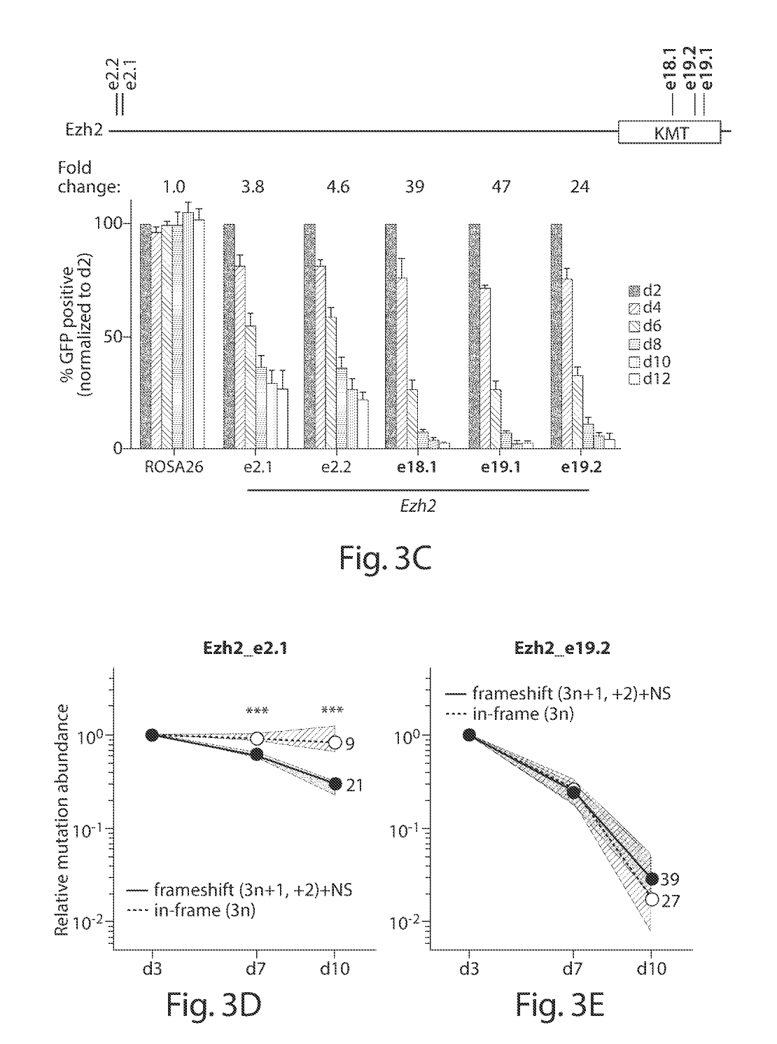

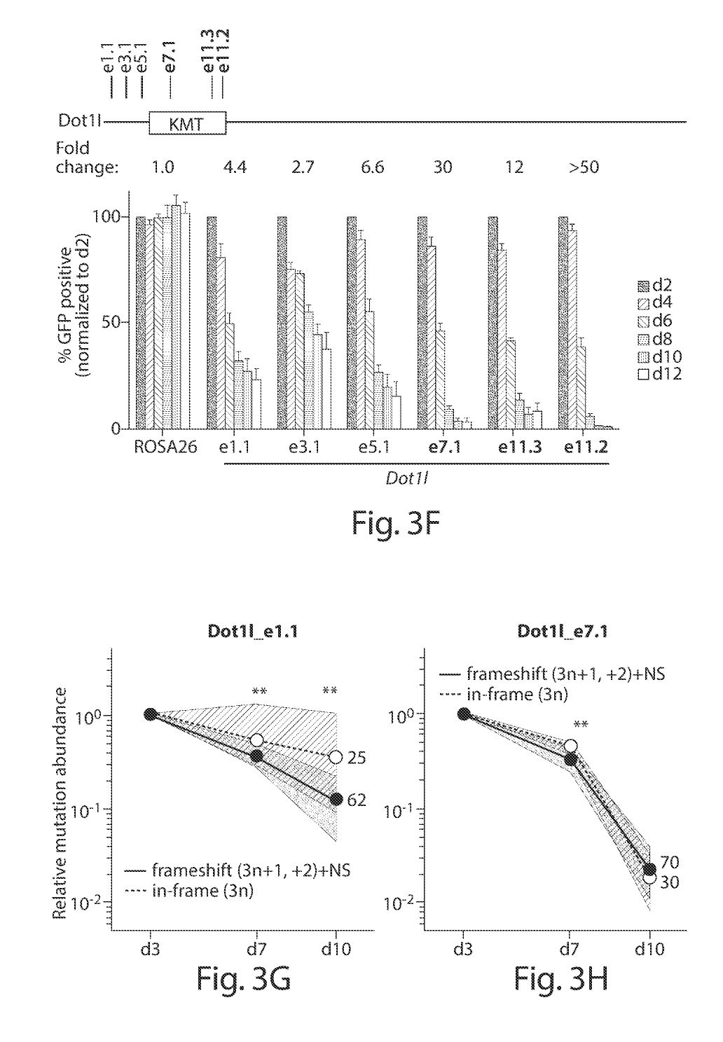

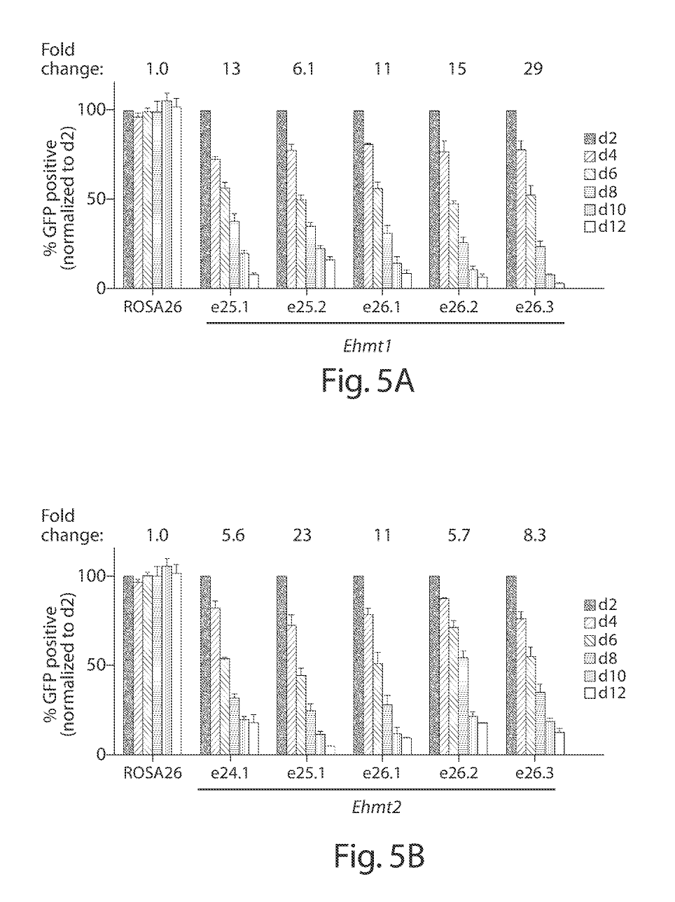

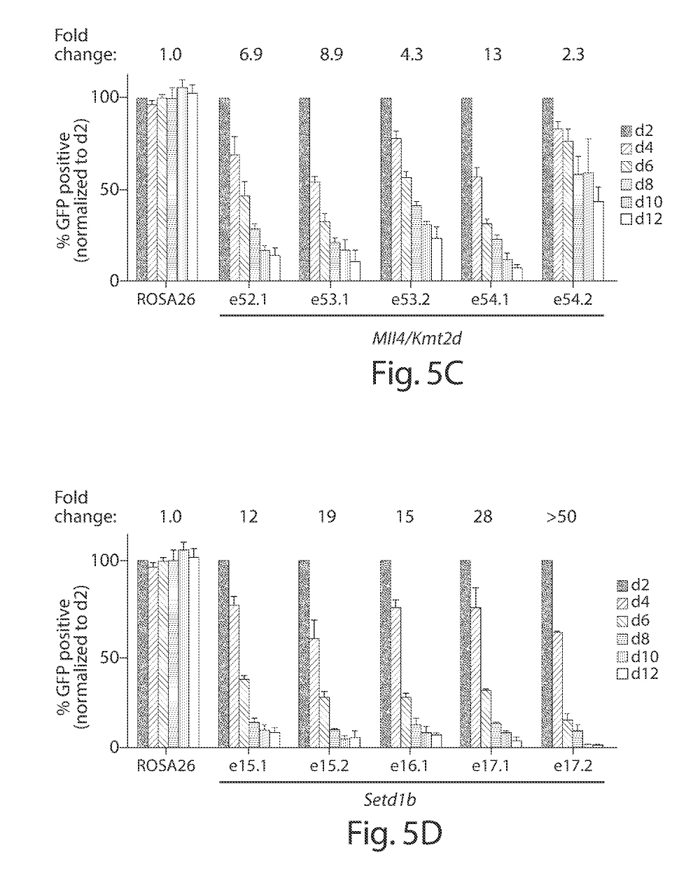

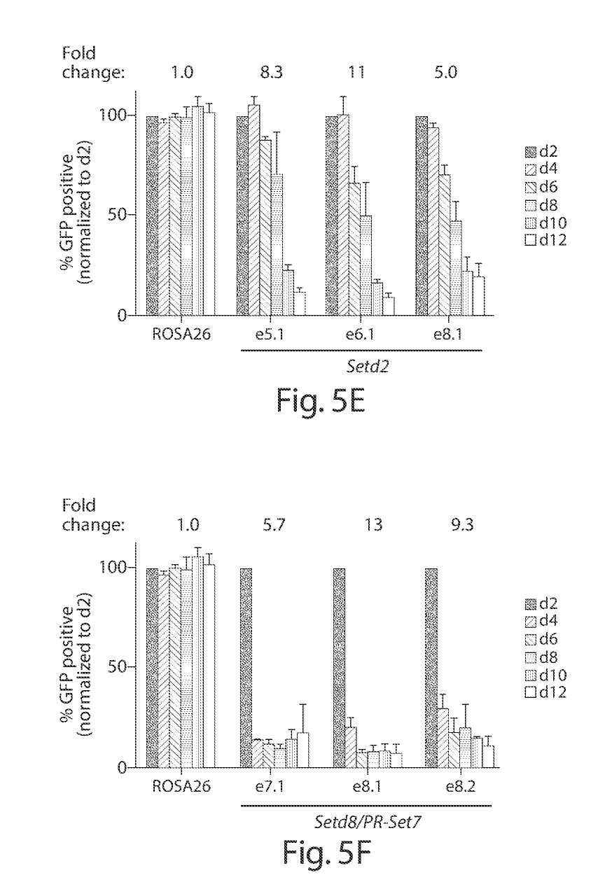

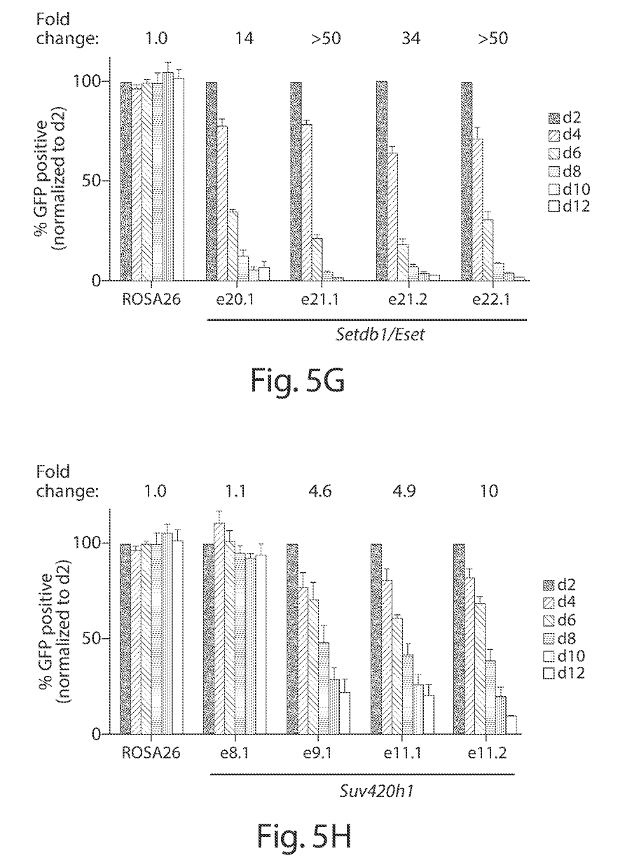

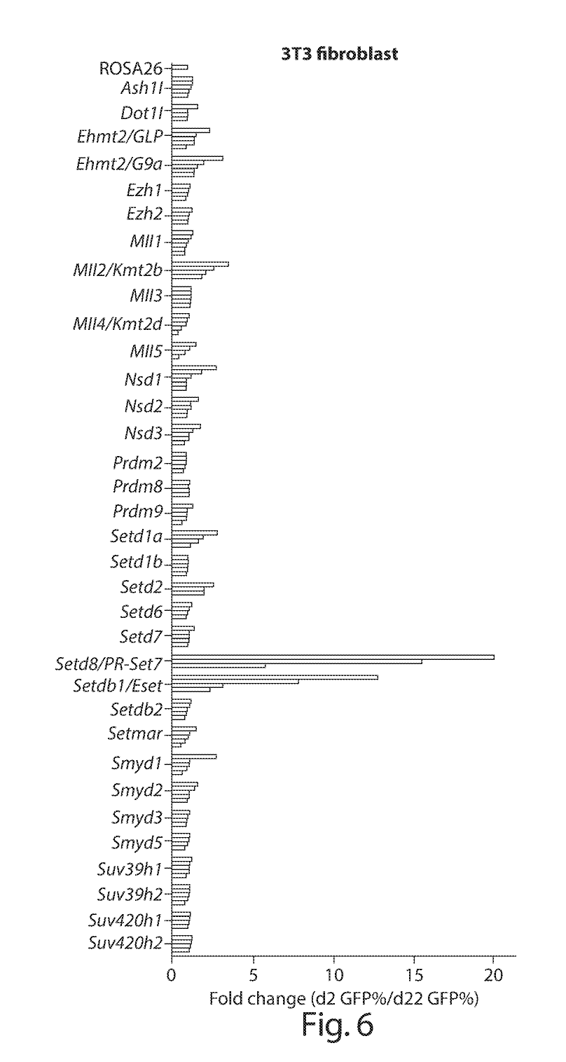

One implication of the experiments described in Examples 1 and 2 is that negative selection CRISPR screens that seek to discover therapeutic targets should utilize sgRNA libraries that target protein domains predicted to be amenable to chemical inhibition. To evaluate this, a sgRNA library was designed to target all of the known lysine methyltransferase (KMT) domains, a target class for which selective small-molecule KMT inhibitors have demonstrated anti-proliferative effects in MLL-AF9 leukemia, such as inhibitors of Dot1l, Ezh2, and Ehmt1/2 (FIG. 3A). These experiments were aimed at determining whether a KMT domain-focused CRISPR screen would identify these known dependencies and, potentially, reveal additional requirements. The impact of ˜150 sgRNAs targeting all 34 KMT domains was evaluated using sgRNA/GFP-depletion assays over 12 days (FIG. 3B). Importantly, Dot11, Ezh2, Ehmt1, and Ehmt2 KMT domain-targeting sgRNAs led to a consistent and pronounced negative selection and were among the top dependencies identified in the screen (FIGS. 3B, 3C, 3F and FIGS. 5A and 5B). In addition, this screen nominated several other KMT domains as being required in MLL-AF9 leukemia, including Setd1b, Setdb1/Eset, and Setd8/PR-Set7, Mll4/Kmt2d, Setd2, and Suv420h1 (FIGS. 5C-5H). A recent study showed that genetic inactivation of Mll4 impairs MLL-AF9-induced leukemia, and the results provided herein suggest that this function is mediated, at least in part, through its KMT domain. By implementing the same sgRNA screen in Cas9⁺ NIH3T3 fibroblasts, only Setdb1 and Setd8 were identified as dependencies in this cell type (FIG. 6), suggesting that many of the KMT requirements in MLL-AF9 leukemia are cell type-specific and, perhaps, therapeutically relevant.

Analogous to Brd4 and Smarca4 CRISPR experiments, sgRNAs targeting the KMT domains of Dot1l and Ezh2 led to stronger negative selection than sgRNAs targeting 5′ exons (FIGS. 3C and 3F). This finding is consistent with the functional importance of these KMT domains and the known sensitivity of MLL-AF9 leukemia cells to Dot1l and Ezh2 KMT inhibitors, and further suggests that the performance of sgRNAs in domain-focused CRISPR screens could be utilized to nominate drug targets in cancer. Finally, the deep sequencing analysis of Ezh2 and Dot1l mutation functionality at KMT and non-KMT locations validated these genes as on-target and validated the critical function of the KMT domain (FIGS. 3D, 3E, 3G and 3H). Collectively, these findings support the capabilities of domain-focused CRISPR screens as a means of cancer drug target identification.

FIGS. 3A-3F show date collected from a lysine methyltransferase (KMT) domain-focused CRISPR screen in MLL-AF9 leukemia validates known drug targets and reveals additional dependencies. FIG. 3A: Table listing the known chemical inhibitors of the indicated KMT proteins and the relevant citation that describes their use in MLL-AF9 leukemia. FIG. 3B: Summary of negative selection experiments with sgRNAs targeting the indicated KMT domains plotted as fold-change of GFP-positivity (d2/d12). Each bar represents the mean value of three independent biological replicates for an independent sgRNA targeting the indicated KMT domain. Red coloring indicates KMT domains for which prior pharmacological validation. A 20-fold cutoff was applied for visualization purposes, and the actual fold-change can be found in FIG. 5. FIGS. 3C and 3E: Negative selection assays for sgRNAs targeting Ezh2 or Dot1l, as described in FIG. 11. sgRNAs targeting the KMT domain are labeled. Fold-change indicates the (d2/d12) GFP-percentage. n=3. FIGS. 3D and 3F: Deep sequencing analysis of mutation abundance for indicated sgRNAs targeting Ezh2 or Dot1l, as described in FIG. 2B-D. All error bars in this figure represent SEM.

References, each of which is incorporated herein
1. Shalem, O. et al. Genome-scale CRISPR-Cas9 knockout screening in human cells. Science 343, 84-87 (2014).
2. Wang, T., Wei, J. J., Sabatini, D. M. & Lander, E. S. Genetic screens in human cells using the CRISPR-Cas9 system. Science 343, 80-84 (2014).
3. Koike-Yusa, H., Li, Y., Tan, E. P., Velasco-Herrera Mdel, C. & Yusa, K. Genome-wide recessive genetic screening in mammalian cells with a lentiviral CRISPR-guide RNA library. Nature biotechnology 32, 267-273 (2014).
4. Zhou, Y. et al. High-throughput screening of a CRISPR/Cas9 library for functional genomics in human cells. Nature 509, 487-491 (2014).
5. Hsu, P. D., Lander, E. S. & Zhang, F. Development and applications of CRISPR-Cas9 for genome engineering. Cell 157, 1262-1278 (2014).
6. Cong, L. et al. Multiplex genome engineering using CRISPR/Cas systems. Science 339, 819-823 (2013).
7. Mali, P. et al. RNA-guided human genome engineering via Cas9. Science 339, 823-826 (2013).
8. Jinek, M. et al. A programmable dual-RNA-guided DNA endonuclease in adaptive bacterial immunity. Science 337, 816-821 (2012).
9. Doench, J. G. et al. Rational design of highly active sgRNAs for CRISPR-Cas9-mediated gene inactivation. Nature biotechnology (2014).
10. Fu, Y. et al. High-frequency off-target mutagenesis induced by CRISPR-Cas nucleases in human cells. Nature biotechnology 31, 822-826 (2013).
11. Hsu, P. D. et al. DNA targeting specificity of RNA-guided Cas9 nucleases. Nature biotechnology 31, 827-832 (2013).
12. Pattanayak, V. et al. High-throughput profiling of off-target DNA cleavage reveals RNA-programmed Cas9 nuclease specificity. Nature biotechnology 31, 839-843 (2013).
13. Zuber, J. et al. RNAi screen identifies Brd4 as a therapeutic target in acute myeloid leukaemia. Nature 478, 524-528 (2011).
14. Zuber, J. et al. Toolkit for evaluating genes required for proliferation and survival using tetracycline-regulated RNAi. Nature biotechnology 29, 79-83 (2011).
15. McJunkin, K. et al. Reversible suppression of an essential gene in adult mice using transgenic RNA interference. Proceedings of the National Academy of Sciences of the United States of America 108, 7113-7118 (2011).
16. Shi, J. et al. Role of SWI/SNF in acute leukemia maintenance and enhancer-mediated Myc regulation. Genes & development 27, 2648-2662 (2013).
17. Wang, E. et al. Histone H2B ubiquitin ligase RNF20 is required for MLL-rearranged leukemia. Proceedings of the National Academy of Sciences of the United States of America 110, 3901-3906 (2013).
18. Shi, J. et al. The Polycomb complex PRC2 supports aberrant self-renewal in a mouse model of MLL-AF9;Nras(G12D) acute myeloid leukemia. Oncogene 32, 930-938 (2013).
19. Mertz, J. A. et al. Targeting MYC dependence in cancer by inhibiting BET bromodomains. Proceedings of the National Academy of Sciences of the United States of America 108, 16669-16674 (2011).
20. Dawson, M. A. et al. Inhibition of BET recruitment to chromatin as an effective treatment for MLL-fusion leukaemia. Nature 478, 529-533 (2011).
21. Findlay, G. M., Boyle, E. A., Hause, R. J., Klein, J. C. & Shendure, J. Saturation editing of genomic regions by multiplex homology-directed repair. Nature 513, 120-123 (2014).
22. Kaelin, W. G., Jr. Molecular biology. Use and abuse of RNAi to study mammalian gene function. Science 337, 421-422 (2012).
23. Lehnertz, B. et al. The methyltransferase G9a regulates HoxA9-dependent transcription in AML. Genes & development 28, 317-327 (2014).
24. Kim, W. et al. Targeted disruption of the EZH2-EED complex inhibits EZH2-dependent cancer. Nature chemical biology 9, 643-650 (2013).
25. Daigle, S. R. et al. Selective killing of mixed lineage leukemia cells by a potent small-molecule DOT1L inhibitor. Cancer cell 20, 53-65 (2011).
26. Xu, B. et al. Selective inhibition of EZH2 and EZH1 enzymatic activity by a small molecule suppresses MLL-rearranged leukemia. Blood (2014).
27. Santos, M. A. et al. DNA-damage-induced differentiation of leukaemic cells as an anti-cancer barrier. Nature 514, 107-111 (2014).
28. Kuscu, C., Arslan, S., Singh, R., Thorpe, J. & Adli, M. Genome-wide analysis reveals characteristics of off-target sites bound by the Cas9 endonuclease. Nature biotechnology 32, 677-683 (2014).
29. Wu, X. et al. Genome-wide binding of the CRISPR endonuclease Cas9 in mammalian cells. Nature biotechnology 32, 670-676 (2014).

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of determining whether a functional domain of a candidate protein is essential for viability of cells of interest, comprising:
(a) introducing, into a population of Cas9-expressing cells of interest, a nucleic acid encoding a single guide RNA (sgRNA) that targets a first region of an gene encoding a candidate protein, wherein the first region encodes a functional domain of the candidate protein, thereby producing a first population of cells comprising a subpopulation of cells that comprise Cas9 nuclease and sgRNA that targets the first region;
(b) introducing, into a population of Cas9-expressing cells of interest, a nucleic acid encoding a sgRNA that targets a second region of a gene encoding the candidate protein, wherein the second region is 5′ to the first region and does not encode a functional domain of the candidate protein, thereby producing a second population of cells comprising subpopulation of cells that comprise Cas9 nuclease and sgRNA that targets the second region;
(c) culturing the first population of cells produced in (a) and the second populations of cells produced in (b) under conditions that result in CRISPR-induced indel mutagenesis of the first region and of the second region, thereby producing a first population of cultured cells and a second population of cultured cells;
(d) assessing the normalized percentage of sgRNA-positive cells (NP) over time in the first population of cultured cells to determine a decrease over time in the NP for the first population of cultured cells;
(e) assessing the NP over time in the second population of cultured cells to determine a decrease over time in the NP for the second population of cultured cells; and
(f) comparing the decrease in NP for the first population (ΔNP1) to the decrease in NP for the second population (ΔNP2), wherein if ΔNP1 is greater than ΔNP2, the functional domain of the candidate protein is essential for viability of cells of interest.

2. The method of claim 1, further comprising
(g) assessing the normalized relative abundance of in-frame mutations generated by CRISPR-induced indel mutagenesis in cells (NRA-IF) over time in the first population of cultured cells to determine a decrease over time in the NRA-IF for the first population of cultured cells;
(h) assessing the NRA-IF over time in the second population of cultured cells to determine a decrease over time in the NRA-IF for the second population of cultured cells; and
(i) comparing the decrease in NRA-IF for the first population (ΔNRA-IF1) to the decrease in NRA-IF for the second population (ΔNRA-IF2), wherein if ΔNRA1 is greater than ΔNRA-IF2, the functional domain of the candidate protein is confirmed to be essential for viability of cells of interest.

3. The method of claim 1 or 2, further comprising
(j) assessing the normalized relative abundance of frameshift/nonsense mutations generated by CRISPR-induced indel mutagenesis in cells (NRA-F/N) over time in the second population of cultured cells to determine a decrease over time in the NRA-F/N for the second population of cultured cells;
(k) assessing the normalized relative abundance of in-frame mutations in cells (NRA-IF) over time in the second population of cultured cells to determine a decrease over time in the NRA-IF for the second population of cultured cells; and
(I) comparing the decrease in NRA-F/N for the second population (ΔNRA-F/N1) to the decrease in NRA-IF for the second population (ΔNRA-IF2), wherein a ΔNRA-F/N1 that is greater than a ΔNRA-IF2 indicates limited occurrence of off-target effects resulting from CRISPR-induced indel mutagenesis.

4. The method of any one of claims 1-3, wherein the Cas9-expressing cells of (a) and (b) further express a reporter protein.

5. The method of any one of claims 1-3, wherein the encoding the sgRNA of (a) and of (b) each further encode a reporter protein.

6. The method of claim 4 or 5, wherein the normalized percentage of sgRNA-positive cells is assessed by assessing the normalized percentage of reporter protein-positive cells.

7. The method of any one of claims 1-6, wherein the cells of interest are cancer cells.

8. The method of any one of claims 1-7, wherein the cells of interest are immune cells.

9. The method of any one of claims 1-8, wherein the Cas9-expressing cells of interest of (a) and of (b) are clonal Cas9⁺ genomically-stable cells derived from the same cell line.

10. The method of any one of claims 1-9, wherein the nucleic acid encoding the sgRNA of (a) and of (b) each is introduced through lentiviral transduction of the Cas9-expressing cells of interest.

11. A method of determining whether a functional domain of a candidate protein is essential for viability of cells of interest, comprising:
(a) introducing, into a subpopulation of a population of Cas9-expressing cells of interest, a nucleic acid encoding a single guide RNA (sgRNA) that targets a first region of a gene encoding a candidate protein, wherein the first region encodes a functional domain of the candidate protein, thereby producing a first population of cells comprising a subpopulation of cells that comprise Cas9nuclease and sgRNA that targets the first region;
(b) introducing, into a subpopulation of a population of Cas9-expressing cells of interest, a nucleic acid encoding a sgRNA that targets a second region of a gene encoding the candidate protein, wherein the second region is 5′ to the first region and does not encode a functional domain of the candidate protein, thereby producing a second population of cells comprising subpopulation of cells that comprise Cas9 nuclease and sgRNA that targets the second region;
(c) culturing the first population of cells produced in (a) and the second populations of cells produced in (b) under conditions that result in CRISPR-induced indel mutagenesis of the first region and of the second region, thereby producing a first population of cultured cells and a second population of cultured cells;
(d) assessing the normalized percentage of CRISPR-induced indel mutations (NP) over time in the first population of cultured cells to determine a decrease over time in the NP for the first population of cultured cells;
(e) assessing the NP over time in the second population of cultured cells to determine a decrease over time in the NP for the second population of cultured cells; and
(f) comparing the decrease in NP for the first population (ΔNP1) to the decrease in NP for the second population (ΔNP2), wherein if ANP1 is greater than ΔNP2, the functional domain of the candidate protein is essential for viability of cells of interest.

12. The method of claim 11, further comprising
(g) assessing the normalized relative abundance of in-frame mutations generated by CRISPR-induced indel mutagenesis in cells (NRA-IF) over time in the first population of cultured cells to determine a decrease over time in the NRA-IF for the first population of cultured cells;
(h) assessing the NRA-IF over time in the second population of cultured cells to determine a decrease over time in the NRA-IF for the second population of cultured cells; and
(i) comparing the decrease in NRA-IF for the first population (ΔNRA-IF1) to the decrease in NRA-IF for the second population (ΔNRA-IF2), wherein if ΔNRA1 is greater than ΔNRA-IF2, the functional domain of the candidate protein is confirmed to be essential for viability of cells of interest.

13. The method of claim 11 or 12, further comprising
(j) assessing the normalized relative abundance of frameshift/nonsense mutations generated by CRISPR-induced indel mutagenesis in cells (NRA-F/N) over time in the second population of cultured cells to determine a decrease over time in the NRA-F/N for the second population of cultured cells;
(k) assessing the normalized relative abundance of in-frame mutations in cells (NRA-IF) over time in the second population of cultured cells to determine a decrease over time in the NRA-IF for the second population of cultured cells; and
(l) comparing the decrease in NRA-F/N for the second population (ΔNRA-F/N1) to the decrease in NRA-IF for the second population (ΔNRA-IF2), wherein a ΔNRA-F/N1 that is greater than a ΔNRA-IF2 indicates limited occurrence of off-target effects resulting from CRISPR-induced indel mutagenesis.

14. The method of any one of claims 11-13, wherein the Cas9-expressing cells of (a) and (b) further express a reporter protein.

15. The method of any one of claims 11-13, wherein the encoding the sgRNA of (a) and of (b) each further encode a reporter protein.

16. The method of claim 14 or 15, wherein the normalized percentage of sgRNA-positive cells is assessed by assessing the normalized percentage of reporter protein-positive cells.

17. The method of any one of claims 11-16, wherein the cells of interest are cancer cells.

18. The method of any one of claims 11-17, wherein the cells of interest are immune cells.

19. The method of any one of claims 11-18, wherein the Cas9-expressing cells of interest of (a) and of (b) are clonal Cas9⁺ genomically-stable cells derived from the same cell line.

20. The method of any one of claims 11-19, wherein the nucleic acid encoding the sgRNA of (a) and of (b) each is introduced through lentiviral transduction of the Cas9-expressing cells of interest.
